(19) United States
(12) Patent Application Publication
(10) Pub. No.: US 2025/0302620 A1
(43) Pub. Date: Oct. 2, 2025

(54) REPLACEMENT HEART VALVE SYSTEM AND METHOD OF LOADING A REPLACEMENT HEART VALVE IMPLANT

(71) Applicant: BOSTON SCIENTIFIC SCIMED, INC., Maple Grove, MN (US)

(72) Inventors: Levi Joel Wolterstorff, Saint Paul, MN (US); James Holmberg, Champlin, MN (US); James M. Anderson, Corcoran, MN (US); Laura McKinley, Minneapolis, MN (US); Christopher Evan Korenczuk, Maple Grove, MN (US); Chenhao Fu, Plymouth, MN (US)

(73) Assignee: BOSTON SCIENTIFIC SCIMED, INC., MAPLE GROVE, MN (US)

(21) Appl. No.: 19/096,870

(22) Filed: Apr. 1, 2025

Related U.S. Application Data
(60) Provisional application No. 63/572,592, filed on Apr. 1, 2024.

(51) Int. Cl. A61F 2/24; A61F 2/00; A61F 2/966

(52) U.S. Cl. CPC A61F 2/2436; A61F 2/0095; A61F 2/2418; A61F 2/9661; A61F 2002/9665; A61F 2220/0033; A61F 2220/0075

(57) ABSTRACT

A replacement heart valve system includes an implant delivery subassembly couplable to an implant delivery system. The subassembly includes a first elongate shaft and a stent holder fixedly attached at a distal end of the first elongate shaft, a replacement heart valve implant, and a distal sheath disposed over a distal portion of the implant with a proximal portion of the implant disposed outside of the distal sheath in a pre-loaded configuration. The subassembly is initially physically separate from the delivery system in the pre-loaded configuration. A method of loading the implant into the replacement heart valve system includes positioning the subassembly adjacent to the delivery system, coupling the subassembly to the delivery system in the pre-loaded configuration, and thereafter shifting a proximal sheath of the delivery system over the proximal portion of the implant.

### CROSS-REFERENCE TO RELATED APPLICATION

This application is a continuation of U.S. Patent Application Ser. No. 63/572,592, filed Apr. 1, 2024, entitled “REPLACEMENT HEART VALVE SYSTEM AND METHOD OF LOADING A REPLACEMENT HEARTVALVE IMPLANT”, which is incorporated by reference herein in its entirety.

### TECHNICAL FIELD

The disclosure relates generally to medical devices and more particularly to medical devices that are adapted for implanting stents and medical devices including a stent component.

### BACKGROUND

A wide variety of intracorporeal medical devices have been developed for medical use including artificial heart valves for repair or replacement of diseased heart valves. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

In one example, a replacement heart valve system may comprise an implant delivery subassembly configured to be coupled to an implant delivery system. The implant delivery subassembly may comprise a first elongate shaft comprising a lumen extending therethrough and a stent holder fixedly attached at a distal end of the first elongate shaft, a replacement heart valve implant detachably engaged with the stent holder, and a distal sheath disposed over a distal portion of the replacement heart valve implant and the stent holder with a proximal portion of the replacement heart valve implant disposed outside of the distal sheath to define a pre-loaded configuration. The distal sheath may be configured to be coupled to a distal end of an inner shaft of the implant delivery system and the first elongate shaft is configured to be coupled to an intermediate tubular member of the implant delivery system disposed about the inner shaft.

In addition, or alternatively, to any example disclosed herein, in the pre-loaded configuration the distal sheath is configured to constrain the distal portion of the replacement heart valve implant in a radially collapsed configuration.

In addition, or alternatively, to any example disclosed herein, the lumen of the first elongate shaft is configured to slidably receive the inner shaft of the implant delivery system.

In addition, or alternatively, to any example disclosed herein, the replacement heart valve implant comprises a plurality of valve leaflets.

In addition, or alternatively, to any example disclosed herein, the implant delivery subassembly is configured to be stored in the pre-loaded configuration in a package devoid of fluid.

In addition, or alternatively, to any example disclosed herein, the implant delivery subassembly is configured to be stored in the pre-loaded configuration in a package filled with a fluid.

In addition, or alternatively, to any example disclosed herein, the implant delivery system comprises a first portion of an attachment assembly disposed at a distal end of the intermediate tubular member, the first portion of the attachment assembly being configured to engage with a second portion of the attachment assembly disposed at a proximal end of the elongate shaft of the implant delivery subassembly.

In addition, or alternatively, to any example disclosed herein, the attachment assembly comprises threads.

In addition, or alternatively, to any example disclosed herein, the attachment assembly comprises a snap lock.

In addition, or alternatively, to any example disclosed herein, the attachment assembly comprises a pin lock.

In addition, or alternatively, to any example disclosed herein, the attachment assembly comprises a twist lock.

In addition, or alternatively, to any example disclosed herein, the attachment assembly is configured to non-pivotably couple the first elongate shaft to the intermediate tubular member.

In addition, or alternatively, to any example disclosed herein, a replacement heart valve system may comprise an implant delivery system comprising an outer tubular member coupled to a proximal sheath, an intermediate tubular member slidably disposed within the outer tubular member, and an inner shaft slidably disposed within the intermediate tubular member. The replacement heart valve system may comprise an implant delivery subassembly comprising a first elongate shaft comprising a lumen extending therethrough and a stent holder fixedly attached at a distal end of the first elongate shaft, a replacement heart valve implant detachably engaged with the stent holder, and a distal sheath disposed over a distal portion of the replacement heart valve implant and the stent holder with a proximal portion of the replacement heart valve implant disposed outside of the distal sheath to define a pre-loaded configuration. The implant delivery subassembly may be initially physically separate from the implant delivery system while the implant delivery subassembly is disposed in the pre-loaded configuration.

In addition, or alternatively, to any example disclosed herein, the implant delivery subassembly is couplable to the implant delivery system while the implant delivery subassembly is disposed in the pre-loaded configuration.

In addition, or alternatively, to any example disclosed herein, the distal sheath is configured to be coupled to a distal end of the inner shaft of the implant delivery system and the first elongate shaft is configured to be coupled to the intermediate tubular member of the implant delivery system.

In addition, or alternatively, to any example disclosed herein, after coupling the implant delivery subassembly to the implant delivery system while in the pre-loaded configuration, the proximal sheath is configured to be shifted distally over the proximal portion of the replacement heart valve implant to define a loaded configuration.

In addition, or alternatively, to any example disclosed herein, a method of loading a replacement heart valve implant into a replacement heart valve system may comprise: positioning an implant delivery subassembly adjacent to an implant delivery system, the implant delivery subassembly comprising a first elongate shaft comprising a lumen extending therethrough and a stent holder fixedly attached at a distal end of the first elongate shaft, a replacement heart valve implant detachably engaged with the stent holder, and a distal sheath disposed over a distal portion of the replacement heart valve implant and the stent holder with a proximal portion of the replacement heart valve implant disposed outside of the distal sheath to define a pre-loaded configuration; coupling the implant delivery subassembly to the implant delivery system in the pre-loaded configuration; and after coupling the implant delivery subassembly to the implant delivery system, shifting a proximal sheath of the implant delivery system distally over the proximal portion of the replacement heart valve implant to define a loaded configuration.

In addition, or alternatively, to any example disclosed herein, coupling the implant delivery subassembly to the implant delivery system comprises coupling the first elongate shaft to an intermediate tubular member of the implant delivery system.

In addition, or alternatively, to any example disclosed herein, coupling the implant delivery subassembly to the implant delivery system comprises extending an inner shaft of the implant delivery system through the first elongate shaft and coupling the distal sheath of implant delivery subassembly to the inner shaft of the implant delivery system.

In addition, or alternatively, to any example disclosed herein, wherein the method further comprises: before coupling the implant delivery subassembly to the implant delivery system, removing the implant delivery subassembly from a package in the pre-loaded configuration.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates selected aspects of a replacement heart valve implant;

FIG. 2 illustrates selected aspects of a replacement heart valve system;

FIGS. 3-4 illustrate selected aspects of an implant delivery subassembly of the replacement heart valve system;

FIGS. 5-6 illustrate selected aspects of the implant delivery subassembly disposed within a package;

FIGS. 7-11 schematically illustrate selected aspects of an attachment assembly associated with the implant delivery subassembly and/or the replacement heart valve system;

FIGS. 12-14 illustrate selected aspects of the replacement heart valve system and a method of loading a replacement heart valve implant into the replacement heart valve system;

FIGS. 15-18 illustrate a proximal loader and selected aspects related to its use;

FIGS. 19-24 illustrate a proximal loader and selected aspects related to its use;

FIGS. 25-27 illustrate a proximal loader and selected aspects related to its use; and

FIGS. 28-32 illustrate a proximal loader and selected aspects related to its use.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

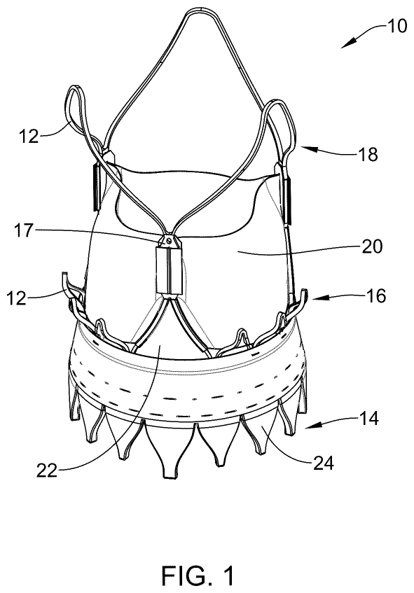

FIG. 1 illustrates selected aspects of a replacement heart valve implant 10. It should be appreciated that the replacement heart valve implant 10 can be any type of replacement heart valve (e.g., a mitral valve, an aortic valve, etc.). Some non-limiting examples of the replacement heart valve implant 10 may include the ACURATE NEO2™, the ACURATE PRIME™, and/or family members thereof from Boston Scientific. Other examples are also contemplated. In use, the replacement heart valve implant 10 may be implanted (e.g., surgically or through transcatheter delivery) in a mammalian heart. The replacement heart valve implant 10 may be configured to allow one-way flow through the replacement heart valve implant 10 from an inflow end to an outflow end.

For the purpose of this disclosure, the discussion herein is directed toward use in treating a native heart valve such as the aortic valve and will be so described in the interest of brevity. This, however, is not intended to be limiting as the skilled person will recognize that the following discussion may also apply to other heart valves, vessels, and/or treatment locations within a patient with no or minimal changes to the structure and/or scope of the disclosure.

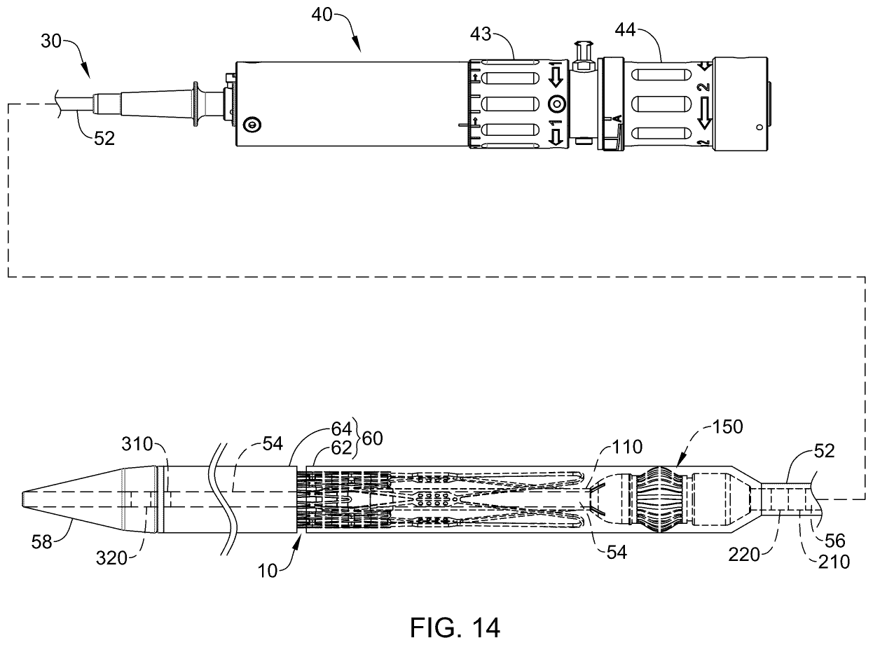

The replacement heart valve implant 10 may include an expandable framework 12 defining a central lumen. In some embodiments, the expandable framework 12 may have a substantially circular cross-section. In some embodiments, the expandable framework 12 can have a non-circular (e.g., D-shaped, elliptical, etc.) cross-section. Some suitable but non-limiting examples of materials that may be used to form the expandable framework 12, including but not limited to metals and metal alloys, composites, ceramics, polymers, and the like, are described below. The expandable framework 12 and/or the replacement heart valve implant 10 may be configured to shift between a radially collapsed configuration (e.g., FIG. 14) and a radially expanded configuration (e.g., FIG. 1). In some embodiments, the expandable framework 12 may be self-expanding from the radially collapsed configuration to the radially expanded configuration. In some embodiments, the expandable framework 12 may be self-biased toward the radially expanded configuration. In some embodiments, the expandable framework 12 may be mechanically expandable from the radially collapsed configuration to the radially expanded configuration. In some embodiments, the expandable framework 12 may be balloon expandable from the radially collapsed configuration to the radially expanded configuration. Other configurations are also contemplated.

In some embodiments, the expandable framework 12 may include and/or define a lower crown 14 proximate and/or at an inflow end, an upper crown 16 proximate and/or at an outflow end, and a plurality of stabilization arches 18 extending downstream of and/or away from the upper crown 16 in a direction opposite the lower crown 14. In some embodiments, the upper crown 16 may be disposed longitudinally and/or axially between the lower crown 14 and the plurality of stabilization arches 18.

In some embodiments, the expandable framework 12 may include and/or define a plurality of commissure posts 17 proximate the outflow end. In some embodiments, the plurality of commissure posts 17 may at least partially define the outflow end. Other configurations are also contemplated. In some embodiments, the plurality of commissure posts 17 may be disposed longitudinally and/or axially between the upper crown 16 and the plurality of stabilization arches 18. In some embodiments, the plurality of stabilization arches 18 may extend downstream of and/or away from the plurality of commissure posts 17 in a direction opposite the lower crown 14. In some embodiments, the upper crown 16 may be disposed longitudinally and/or axially between the lower crown 14 and the plurality of commissure posts 17.

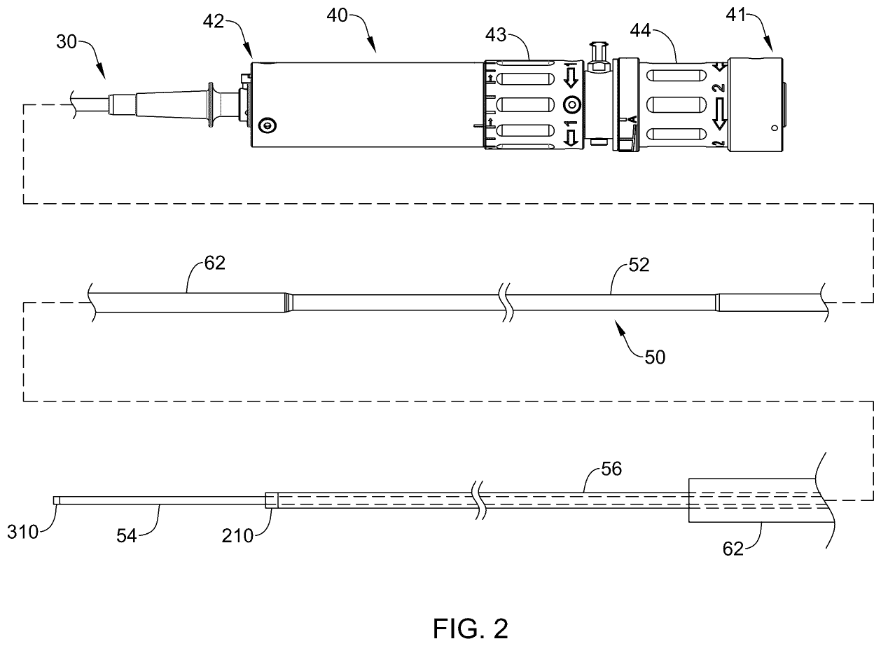

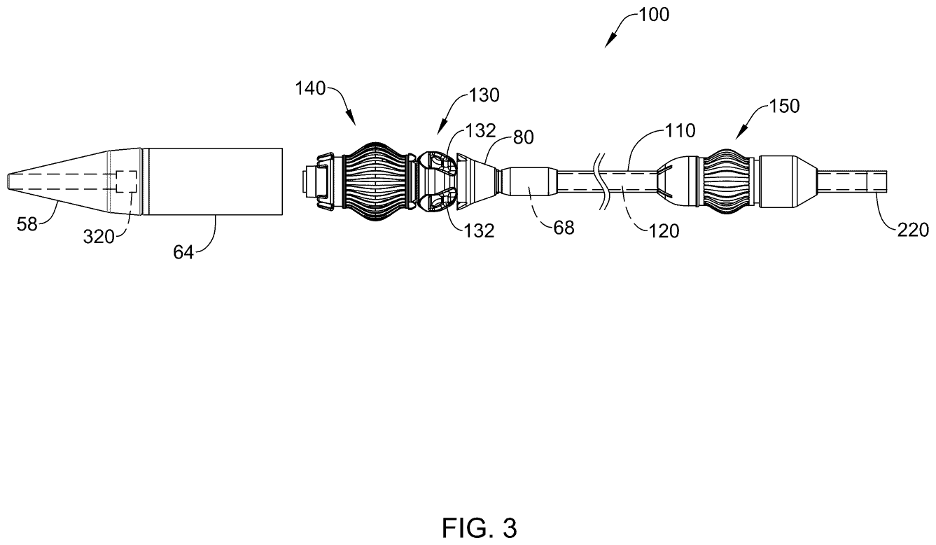

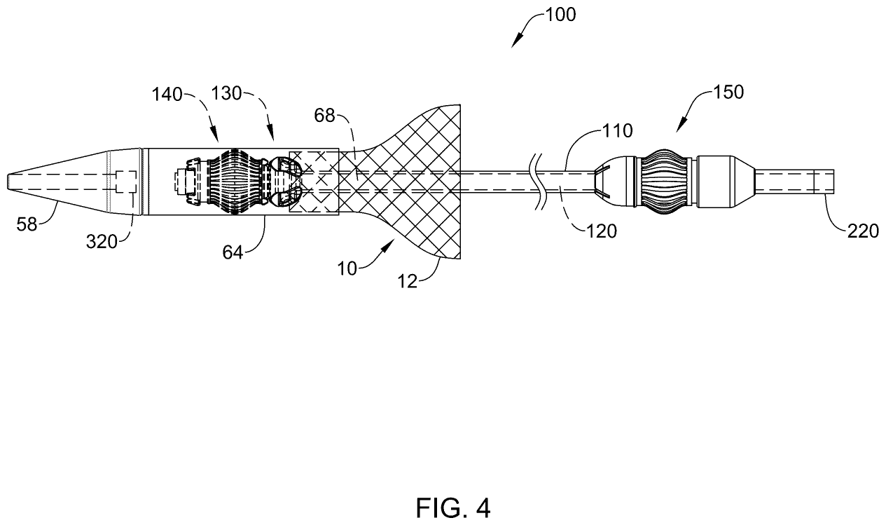

In some embodiments, the replacement heart valve implant 10 may include a proximal portion and a distal portion. In some embodiments, orientation of the replacement heart valve implant 10 may be related to an implant delivery system 30 (e.g., FIG. 2) and/or an implant delivery subassembly 100 (e.g., FIGS. 3-4), and/or a direction of implantation relative to a treatment site (e.g., a native heart valve, the aortic valve, etc.). In some embodiments, the proximal portion may include the outflow end and/or the plurality of stabilization arches 18. In some embodiments, the proximal portion may include the plurality of commissure posts 17, the upper crown 16, and/or the plurality of valve leaflets 20. In some embodiments, the distal portion may include the inflow end and/or the lower crown 14. Other configurations are also contemplated.

In some embodiments, the replacement heart valve implant 10 may include a plurality of valve leaflets 20 disposed within the central lumen. The plurality of valve leaflets 20 may be coupled, secured, and/or fixedly attached to the expandable framework 12. In at least some embodiments, the plurality of valve leaflets 20 may be coupled, secured, and/or fixedly attached to the expandable framework 12 at the plurality of commissure posts 17 to form and/or define a plurality of commissures.

Each of the plurality of valve leaflets 20 may include a root edge coupled to the expandable framework 12 and a free edge (e.g., a coaptation edge) movable relative to the root edge to coapt with the free edges of the other valve leaflets along a coaptation region. In some embodiments, the plurality of valve leaflets 20 can be integrally formed with each other, such that the plurality of valve leaflets 20 is formed as a single unitary and/or monolithic unit. In some embodiments, the plurality of valve leaflets 20 may be formed integrally with other structures such as an inner skirt 22 and/or an outer skirt 24, base structures, liners, or the like.

The plurality of valve leaflets 20 may be configured to shift between an open position and a closed position. The plurality of valve leaflets 20 may be configured to substantially restrict fluid from flowing through the replacement heart valve implant 10 in the closed position. For example, in some embodiments, the free edges of the plurality of valve leaflets 20 may move into coaptation with one another in the closed position to substantially restrict fluid from flowing through the replacement heart valve implant 10. The free edges of the plurality of valve leaflets 20 may be moved apart from each other in the open position to permit fluid flow through the replacement heart valve implant 10 and/or the central lumen. In FIG. 1, the plurality of valve leaflets 20 is shown in the open position or in a partially open position (e.g., a neutral position) that the plurality of valve leaflets 20 may move to when unbiased by fluid flow.

In some embodiments, the plurality of valve leaflets 20 may be comprised of a polymer, such as a thermoplastic polymer. In some embodiments, the plurality of valve leaflets 20 may include at least 50 percent by weight of a polymer. In some embodiments, the plurality of valve leaflets 20 may be formed from porcine pericardium, bovine pericardium or other tissue. Other configurations and/or materials are also contemplated.

In some embodiments, the replacement heart valve implant 10 may include an inner skirt 22 disposed on and/or extending along an inner surface of the expandable framework 12. In at least some embodiments, the inner skirt 22 may be fixedly attached to the expandable framework 12. The inner skirt 22 may direct fluid, such as blood, flowing through the replacement heart valve implant 10 toward the plurality of valve leaflets 20. In at least some embodiments, the inner skirt 22 may be fixedly attached to and/or integrally formed with the plurality of valve leaflets 20. The inner skirt 22 may ensure the fluid flows through the central lumen of the replacement heart valve implant 10 and does not flow around the plurality of valve leaflets 20 when they are in the closed position.

In some embodiments, the replacement heart valve implant 10 can include an outer skirt 24 disposed on and/or extending along an outer surface of the expandable framework 12. In some embodiments, the outer skirt 24 may be disposed at and/or adjacent the lower crown 14. The outer skirt 24 may ensure the fluid flows through the replacement heart valve implant 10 and does not flow around the replacement heart valve implant 10 (e.g., between the expandable framework 12 and the vessel wall).

In some embodiments, the inner skirt 22 and/or the outer skirt 24 may include a polymer, such as a thermoplastic polymer. In some embodiments, the inner skirt 22 and/or the outer skirt 24 may include at least 50 percent by weight of a polymer. In some embodiments, one or more of the plurality of valve leaflets 20, the inner skirt 22, and/or the outer skirt 24 may be formed of the same polymer or polymers. In some embodiments, the polymer may be a polyurethane. In some embodiments, the inner skirt 22 and/or the outer skirt 24 may be substantially impervious to fluid. In some embodiments, the inner skirt 22 and/or the outer skirt 24 may be formed from a thin tissue (e.g., porcine pericardium, bovine pericardium, etc.), a coated fabric material, and/or a nonporous and/or impermeable fabric material. Other configurations are also contemplated. Some suitable but non-limiting examples of materials that may be used to form the inner skirt 22 and/or the outer skirt 24 including but not limited to polymers, composites, and the like, are described below.

In some embodiments, the inner skirt 22 and/or the outer skirt 24 may seal one of, some of, a plurality of, or each of a plurality of interstices formed in the expandable framework 12. In at least some embodiments, sealing the interstices may be considered to prevent fluid from flowing through the interstices of the expandable framework 12. In some embodiments, the inner skirt 22 and/or the outer skirt 24 may be attached to the expandable framework 12 using one or more methods including but not limited to tying with sutures or filaments, adhesive bonding, melt bonding, embedding or over molding, welding, etc.

In some embodiments, the expandable framework 12 and/or the replacement heart valve implant 10 may have an outer extent of about 23 millimeters (mm) (0.905 inches), about 25 mm (0.984 inches), about 27 mm (1.063 inches), about 30 mm (1.181 inches), etc. in an unconstrained configuration (e.g., in the radially expanded configuration). In some embodiments, the expandable framework 12 and/or the replacement heart valve implant 10 may have an outer extent of about 10 mm (0.394 inches), about 9 mm (0.354 inches) about 8 mm (0.315 inches), about 7 mm (0.276 inches), about 6 mm (0.236 inches), etc. in the radially collapsed configuration. Other configurations are also contemplated.

FIG. 2 illustrates selected aspects of a replacement heart valve system, which may include a replacement heart valve implant (not shown) and an implant delivery system 30 for delivering a replacement heart valve implant to a native heart valve (e.g., the aortic valve). The implant delivery system 30 may be compatible with and/or usable with the replacement heart valve implant 10. In FIG. 2, some elements that would be hidden from view are shown in phantom to show relative positioning. It should also be noted that FIG. 2 includes at least one change of scale (e.g., all parts of the figure are not drawn to the same scale) to improve viewability of selected aspects of the implant delivery system 30.

The implant delivery system 30 may include a handle assembly 40 and an elongate shaft assembly 50 extending distally from the handle assembly 40. The handle assembly 40 may include a first end 41 and a second end 42 opposite the first end 41. The elongate shaft assembly 50 may extend distally from the second end 42 of the handle assembly 40. The handle assembly 40 may include one or more rotatable knobs. In some embodiments, the one or more rotatable knobs may include a first rotatable knob 43 and a second rotatable knob 44. In at least some embodiments, the first rotatable knob 43 and/or the second rotatable knob 44 may be configured to rotate about a central longitudinal axis of the implant delivery system 30 and/or the handle assembly 40. Other handle assembly configurations are also contemplated.

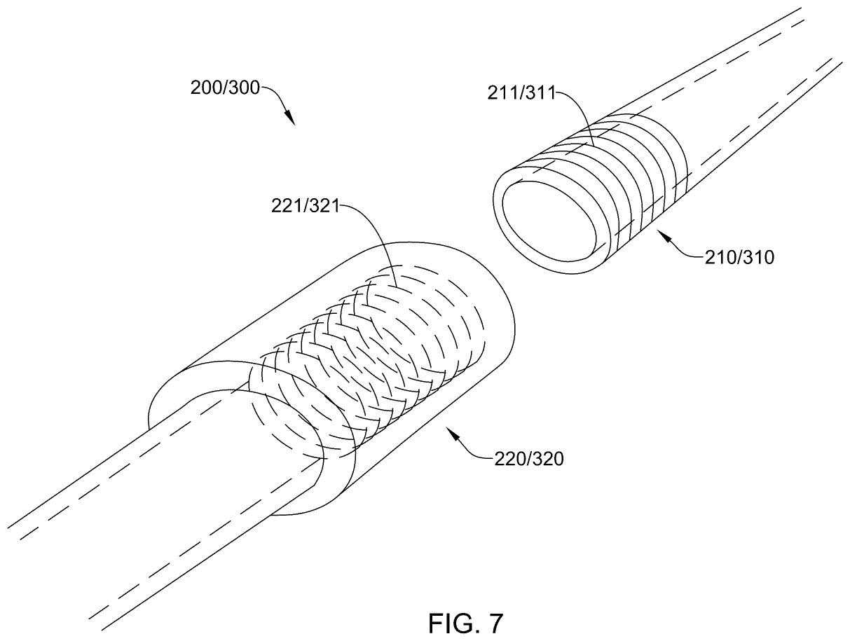

The implant delivery system 30 and/or the elongate shaft assembly 50 may comprise an outer tubular member 52 coupled to a proximal sheath 62. In some embodiments, the proximal sheath 62 may be fixedly attached to the outer tubular member 52. In some embodiments, the proximal sheath 62 may extend distally from a distal end of the outer tubular member 52. In at least some embodiments, the proximal sheath 62 may be configured to constrain at least a portion of the replacement heart valve implant 10 (e.g., FIG. 14) in the radially collapsed configuration. The implant delivery system 30 may comprise an intermediate tubular member 56 slidably disposed within the outer tubular member 52. The implant delivery system 30 may comprise a first portion 210 of an attachment assembly 200 (e.g., FIGS. 7-14) disposed at a distal end of the intermediate tubular member 56. The implant delivery system 30 may comprise an inner shaft 54 slidably disposed within the intermediate tubular member 56. In some embodiments, the implant delivery system 30 may comprise a first portion 310 of an attachment assembly 300 (e.g., FIGS. 7-14) disposed at a distal end of the inner shaft 54. In some embodiments, the inner shaft 54 may include and/or at least partially define a lumen extending therethrough. In some embodiments, the lumen may extend through the handle assembly 40. In at least some embodiments, the lumen may be configured to slidably receive a guidewire therein.

In at least some embodiments, the intermediate tubular member 56 may be axially secured to the handle assembly 40. In some embodiments, the inner shaft 54 and/or the outer tubular member 52 may be coupled to and/or axially secured to the handle assembly 40. In the context of this disclosure, “axially secured” (and/or variants thereof) generally means that the feature or element is not free floating in an axial direction relative to another feature or element. For example, the feature or element may not be permitted to move freely in an axial direction or on its own but in some embodiments may be movable in an axial direction using a means or mechanism for controlled movement. In some embodiments, the intermediate tubular member 56 may be fixedly secured to the handle assembly 40.

In some embodiments, the outer tubular member 52 may be axially movable relative to the intermediate tubular member 56 via actuation of the handle assembly 40 and/or a portion thereof. In some embodiments, the inner shaft 54 may be axially movable relative to the intermediate tubular member 56 via actuation of the handle assembly 40 and/or a portion thereof. In some embodiments, the outer tubular member 52 and the inner shaft 54 may be axially movable relative to each other via actuation of the handle assembly 40 and/or a portion thereof. In at least some embodiments, the inner shaft 54 and the outer tubular member 52 are each axially translatable relative to the intermediate tubular member 56 independently of each other. For example, the inner shaft 54 may be translated relative to the intermediate tubular member 56 without translating the outer tubular member 52 relative to the intermediate tubular member 56, and vice versa.

In at least some embodiments, the proximal sheath 62 may have an outer diameter greater than an outer diameter of the outer tubular member 52. In some embodiments, the proximal sheath 62 may have an inner diameter greater than the outer diameter of the outer tubular member 52. In at least some embodiments, the proximal sheath 62 may form at least a portion of an implant holding portion 60 (e.g., FIG. 14) of the replacement heart valve system.

FIGS. 3-4 illustrate selected aspects of an implant delivery subassembly 100 associated with and/or compatible with the implant delivery system 30 described herein. In some embodiments, the implant delivery subassembly 100 may be configured to be coupled to the implant delivery system 30. In some embodiments, the implant delivery subassembly 100 may comprise a first elongate shaft 110 comprising a lumen 120 extending therethrough. In some embodiments, the lumen 120 may extend completely through the first elongate shaft 110. In some embodiments, the lumen 120 may be sized and configured to slidably receive the inner shaft 54 of the implant delivery system 30 therein. In at least some embodiments, the first elongate shaft 110 may be similar in diameter, shape, and/or construction to the intermediate tubular member 56 (e.g., FIG. 2).

The implant delivery subassembly 100 may comprise a second portion 220 of the attachment assembly 200 (e.g., FIGS. 7-14) disposed at a proximal end of the first elongate shaft 110. The first portion 210 of the attachment assembly 200 may be configured to engage with the second portion 220 of the attachment assembly 200. In at least some embodiments, the attachment assembly 200 (e.g., FIGS. 7-14) may be configured to non-pivotably couple the first elongate shaft 110 to the intermediate tubular member 56. Some non-limiting exemplary configurations of the attachment assembly 200 are discussed in more detail below.

In some embodiments, the first elongate shaft 110 may comprise a stent holder 130 fixedly attached proximate and/or at a distal end of the first elongate shaft 110. In some alternative embodiments, the stent holder 130 may be integrally formed with the first elongate shaft 110. In some embodiments, the stent holder 130 may include a body, a first end portion extending proximally from the body, and a second end portion disposed opposite the first end portion. In some embodiments, at least a portion of the first end portion may extend radially outward from and/or radially outward of the body. In some embodiments, the first end portion may have a generally bulbous shape. In some embodiments, the stent holder 130 may be configured and/or adapted to be visible under fluoroscopy. In some embodiments, the stent holder 130 may be formed from stainless steel. Some suitable but non-limiting materials for the stent holder 130 and/or components or elements thereof are described below.

In some embodiments, the implant delivery subassembly 100 may comprise the replacement heart valve implant 10 (e.g., FIG. 4). The replacement heart valve implant 10 may be configured to detachably engage with the stent holder 130. In some embodiments, the replacement heart valve implant 10 may be configured to detachably engage with the stent holder 130 in the radially collapsed configuration.

In some embodiments, the implant delivery subassembly 100 may comprise a distal sheath 64. The distal sheath 64 may be fixedly attached to a distal tip 58. The distal tip 58 may be tapered radially inward in a distal direction. The distal sheath 64 may extend proximally from the distal tip 58. In some embodiments, the distal sheath 64 may be configured to be disposed over a distal portion of the replacement heart valve implant 10 and the stent holder 130 with a proximal portion of the replacement heart valve implant 10 disposed outside of the distal sheath 64 to define a pre-loaded configuration, shown in FIG. 4. In some embodiments, in the pre-loaded configuration, the distal sheath 64 may be disposed over the distal portion of the replacement heart valve implant 10 to detachably engage the distal portion of the replacement heart valve implant 10 with the stent holder 130 in the radially collapsed configuration with a proximal portion of the replacement heart valve implant 10 disposed outside of the distal sheath 64 in the radially expanded configuration (or at least partially disposed in the radially expanded configuration). In at least some embodiments, in the pre-loaded configuration, the distal sheath 64 may be configured to constrain the distal portion of the replacement heart valve implant 10 in the radially collapsed configuration.

The implant delivery subassembly 100, the distal tip 58, and/or the distal sheath 64 may comprise a second portion 320 of the attachment assembly 300 (e.g., FIGS. 7-13). The first portion 310 of the attachment assembly 300 may be configured to engage with the second portion 320 of the attachment assembly 300. In at least some embodiments, the attachment assembly 300 (e.g., FIGS. 7-13) may be configured to non-pivotably couple the inner shaft 54 to the distal tip 58 and/or the distal sheath 64. Some non-limiting exemplary configurations of the attachment assembly 300 are discussed in more detail below.

In some embodiments, the stent holder 130 may be configured to engage the replacement heart valve implant 10 and/or the expandable framework 12 in the radially collapsed configuration and/or when the distal portion of the replacement heart valve implant 10 is constrained within the distal sheath 64, as seen in FIG. 4. In some embodiments, the stent holder 130 may include at least one projection 132 (e.g., FIG. 3) configured to engage the expandable framework 12 in the radially collapsed configuration. In some embodiments, the at least one projection 132 may be configured to engage the inflow end of the replacement heart valve implant 10 and/or the expandable framework 12 in the radially collapsed configuration. In some embodiments, the at least one projection 132 may extend into and/or through interstices of the expandable framework 12. In some embodiments, the replacement heart valve implant 10 and/or the expandable framework 12 may include at least one mounting loop configured to receive and/or engage with the at least one projection 132. Other configurations are also contemplated.

In some embodiments, the implant delivery subassembly 100 and/or the first elongate shaft 110 may comprise a distal shuttle assembly 140 disposed on the first elongate shaft 110. The distal shuttle assembly 140 may be disposed on the first elongate shaft 110 distal of the replacement heart valve implant 10 when the distal portion of the replacement heart valve implant 10 is constrained within the distal sheath 64 in the radially collapsed configuration. In at least some embodiments, the distal shuttle assembly 140 may be fixedly attached to the first elongate shaft 110 proximate and/or at the distal end of the first elongate shaft 110. In some embodiments, the distal shuttle assembly 140 may be configured to center the first elongate shaft 110 within the distal sheath 64 during closure of the distal sheath 64 over the distal portion of the replacement heart valve implant 10 (e.g., during pre-loading of the replacement heart valve implant 10). In some embodiments, the distal shuttle assembly 140 may include a plurality of spring elements self-biased to expand radially outward from the first elongate shaft 110, thereby exerting a radially outward force against an inner surface of the distal sheath 64 during loading of the replacement heart valve implant 10. In some embodiments, a proximal portion of the distal shuttle assembly 140 may be engaged with a distal portion of the stent holder 130. In some embodiments, the proximal portion of the distal shuttle assembly 140 may be disposed radially outward of and/or may surround the distal portion of the stent holder 130. In some embodiments, the distal shuttle assembly 140 may include the stent holder 130. Other configurations are also contemplated.

In some embodiments, the implant delivery subassembly 100 and/or the first elongate shaft 110 may include a primary visual indicator 68 (e.g., FIG. 3) disposed within the replacement heart valve implant 10 when the distal portion of the replacement heart valve implant 10 is constrained within the distal sheath 64 in the radially collapsed configuration. The primary visual indicator 68 may be configured and/or adapted to be visible under fluoroscopy with an imaging device. Other imaging means suitable for use with transcatheter surgical procedures are also contemplated. The implant delivery subassembly 100 and/or the primary visual indicator 68 may be configured to cooperate with the imaging device to position the replacement heart valve implant 10 at a desired insertion depth within the native heart valve (e.g., the aortic valve). In some embodiments, the primary visual indicator 68 may be and/or may include a marker band. In some embodiments, the primary visual indicator 68 may be at least partially radiopaque. In some embodiments, the primary visual indicator 68 may be completely radiopaque. Other configurations are also contemplated.

In some embodiments, the implant delivery subassembly 100 may include an atraumatic transition shield 80 (e.g., FIG. 3). The atraumatic transition shield 80 may be disposed adjacent the stent holder 130. In some embodiments, the atraumatic transition shield 80 may be disposed between the stent holder 130 and the second portion 220 of the attachment assembly 200. In some embodiments, the atraumatic transition shield 80 may be disposed proximal of the stent holder 130. In some embodiments, the atraumatic transition shield 80 may be disposed at and/or adjacent the first end portion of the stent holder 130. In some embodiments, the atraumatic transition shield 80 may axially overlap the first end portion of the stent holder 130. In some embodiments, the atraumatic transition shield 80 may be disposed radially outward of at least a portion of the first end portion of the stent holder 130. In some embodiments, the atraumatic transition shield 80 may be tapered radially inward in the downstream direction and/or the proximal direction and/or toward the handle assembly 40. The atraumatic transition shield 80 may be configured to prevent the replacement heart valve implant 10, the expandable framework 12, the plurality of valve leaflets 20, etc. from catching on the stent holder 130 as the implant delivery system 30 is withdrawn after deploying the replacement heart valve implant 10 within the native heart valve (e.g., the aortic valve).

In some embodiments, the primary visual indicator 68 may be disposed adjacent a proximal end of the atraumatic transition shield 80. In some embodiments, the primary visual indicator 68 may be disposed proximal of the atraumatic transition shield 80. In some embodiments, the primary visual indicator 68 and the atraumatic transition shield 80 may axially overlap. In some embodiments, the primary visual indicator 68 may be fixedly attached to the first elongate shaft 110. In some embodiments, the primary visual indicator 68 may be embedded in the first elongate shaft 110. In some embodiments, the primary visual indicator 68 may be secured and/or fixedly attached to the first elongate shaft 110, for example by adhesive bonding, welding, shrink wrap, etc. Other configurations are also contemplated.

In some embodiments, the implant delivery subassembly 100 and/or the first elongate shaft 110 may comprise a proximal shuttle assembly 150 disposed on the first elongate shaft 110 proximal of the replacement heart valve implant 10 when the distal portion of the replacement heart valve implant 10 is constrained within the distal sheath 64 in the radially collapsed configuration. In some embodiments, the proximal shuttle assembly 150 may be configured to slide along the first elongate shaft 110. In some embodiments, the proximal shuttle assembly 150 may be fixedly attached to the first elongate shaft 110. In some embodiments, the proximal shuttle assembly 150 may be configured to center the first elongate shaft 110 within the proximal sheath 62 (e.g., FIG. 2, FIG. 14) during loading of the replacement heart valve implant 10 into the implant delivery system 30 and/or the implant holding portion 60 (e.g., FIG. 14).

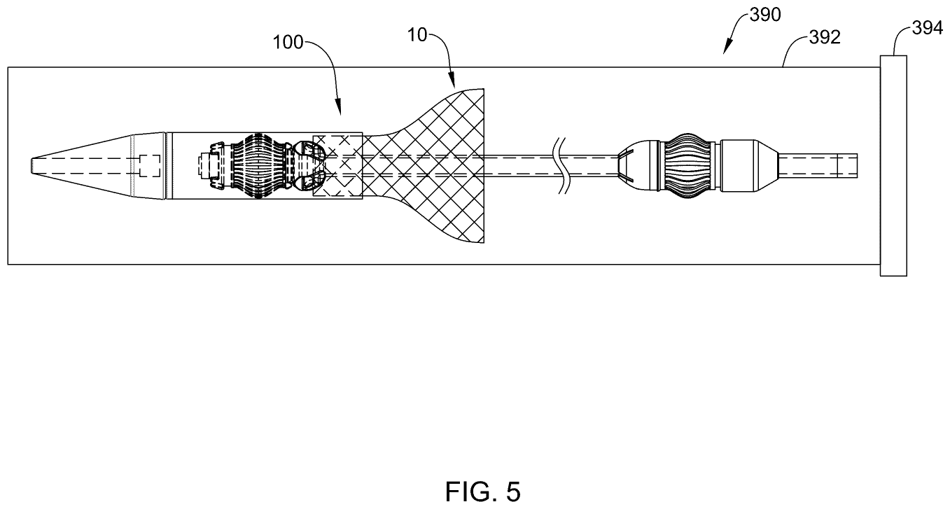

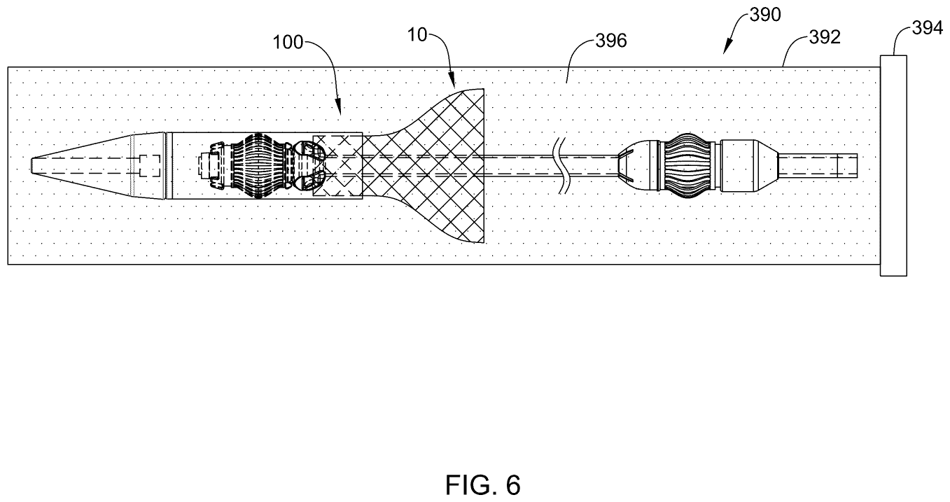

FIGS. 5-6 schematically illustrate selected aspects of the replacement heart valve system of the disclosure. In some embodiments, the implant delivery subassembly 100 of FIG. 4 may be configured to be stored in the pre-loaded configuration in a package 390. In at least some embodiments, the package 390 may be sealed at and/or by the manufacturer with the implant delivery subassembly 100 disposed therein in the pre-loaded configuration. As such, the end user (e.g., a practitioner, surgeon, etc.) would not need to load the distal portion of the replacement heart valve implant 10 into the distal sheath 64 prior to use. In some embodiments, the package 390 may comprise a package body 392 and a cap 394. In some embodiments, the cap 394 may engage the package body 392 via threads. In some embodiments, the cap 394 may snap on to the package body 392. Other configurations are also contemplated. In some embodiments, the package 390 may comprise the package body 392 and a cover (not shown) sealingly attached to the package body 392. In one non-limiting example, the cover may be configured to peel away from the package body 392 when pulled on. Other configurations are also contemplated. In some embodiments, the package 390 may be adapted and configured as a single-use container, such that once the package 390 has been opened, the package 390 cannot be re-sealed. In some alternative embodiments, the package 390 may be reusable with appropriate sterilization.

In some embodiments, the implant delivery subassembly 100 of FIG. 4 may be configured to be stored in the pre-loaded configuration in a package 390 devoid of fluid, as seen in FIG. 5. In some embodiments, the replacement heart valve implant 10 may be stored within the package 390 in a dry state. Accordingly, in some embodiments, the plurality of valve leaflets 20, and/or other elements or components of the replacement heart valve implant 10, may be stored and/or packaged in a dry state. In some embodiments, the replacement heart valve implant 10 may be sterilized prior to disposing the replacement heart valve implant 10 within the package 390. In some alternative embodiments, the replacement heart valve implant 10 may be unsterilized prior to disposing the replacement heart valve implant 10 within the package 390. In one non-limiting example, the replacement heart valve implant 10 may be sterilized within the package 390. In another non-limiting example, the replacement heart valve implant 10 may be sterilized after opening the package 390 within the operating room or a sterile preparatory room adjoining the operating room. In another non-limiting example, the replacement heart valve implant 10 may be sterilized after removing the replacement heart valve implant 10 from the package 390.

In some embodiments, the implant delivery subassembly 100 of FIG. 4 may be configured to be stored in the pre-loaded configuration in the package 390 filled with a fluid 396, as seen in FIG. 6. In some embodiments, the replacement heart valve implant 10 may be stored within the package 390 a wet state. Accordingly, in some embodiments, the plurality of valve leaflets 20, and/or other elements or components of the replacement heart valve implant 10, may be stored and/or packaged in a wet state. In some embodiments, the fluid 396 may be a storage fluid configured to preserve the replacement heart valve implant 10 and/or components thereof (e.g., the plurality of leaflets, etc.). In some embodiments, the replacement heart valve implant 10 may be sterilized prior to disposing the replacement heart valve implant 10 within the package 390. In some alternative embodiments, the replacement heart valve implant 10 may be unsterilized prior to disposing the replacement heart valve implant 10 within the package 390. In one non-limiting example, the replacement heart valve implant 10 may be sterilized within the package 390. In some embodiments, the fluid 396 may be a sterilization fluid. In some embodiments, the fluid 396 may be glutaraldehyde. Other suitable fluids and/or types of fluids are also contemplated. In another non-limiting example, the replacement heart valve implant 10 may be sterilized after opening the package 390 within the operating room or a sterile preparatory room adjoining the operating room. In another non-limiting example, the replacement heart valve implant 10 may be sterilized after removing the replacement heart valve implant 10 from the package 390.

FIGS. 7-11 schematically illustrate selected aspects of the attachment assembly 200 and/or the attachment assembly 300, and/or some exemplary configurations thereof. In at least some embodiments, the attachment assembly 200 and the attachment assembly 300 may be the same, may be different, and/or may be interchanged. In some embodiments, the attachment assembly 200 may have the same construction and/or characteristics as the attachment assembly 300. In some embodiments, the attachment assembly 200 may have different construction and/or characteristics than the attachment assembly 300. As discussed herein, the attachment assembly 200 may comprise a first portion 210 disposed at the distal end of the intermediate tubular member 56 and a second portion 220 disposed at the proximal end of the first elongate shaft 110. Similarly, the attachment assembly 300 may comprise a first portion 310 disposed at the distal end of the inner shaft 54 and a second portion 320 disposed within the distal tip 58 and/or the distal sheath 64.

In some embodiments, the first portion 210 and the second portion 220 of the attachment assembly 200 may be configured to be engaged with each other and/or may be configured to be coupled together while the implant delivery subassembly 100 is in the pre-loaded configuration. In one non-limiting example, the first portion 210 and the second portion 220 of the attachment assembly 200 may be configured to be engaged with each other and/or may be configured to be coupled together to couple the first elongate shaft 110 of the implant delivery subassembly 100 to the intermediate tubular member 56 of the implant delivery system 30 by a practitioner prior to use (e.g., in the operating room or a sterile preparatory room adjoining the operating room).

In some embodiments, the first portion 310 and the second portion 320 of the attachment assembly 300 may be configured to be engaged with each other and/or may be configured to be coupled together while the implant delivery subassembly 100 is in the pre-loaded configuration. In one non-limiting example, the first portion 310 and the second portion 320 of the attachment assembly 300 may be configured to be engaged with each other and/or may be configured to be coupled together to couple the distal tip 58 and/or the distal sheath 64 of the implant delivery subassembly 100 to the inner shaft 54 of the implant delivery system 30 by a practitioner prior to use (e.g., in the operating room or a sterile preparatory room adjoining the operating room).

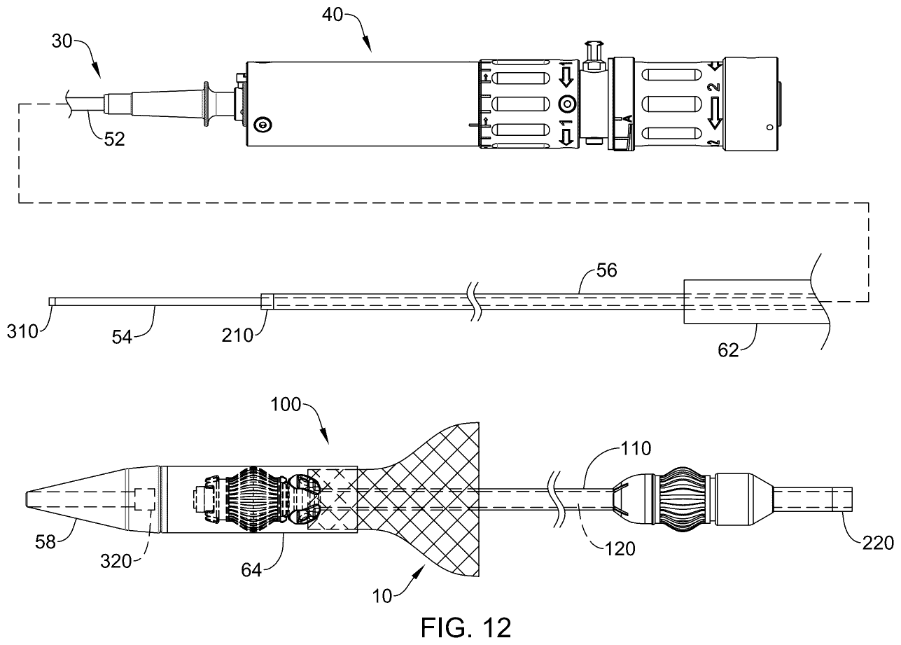

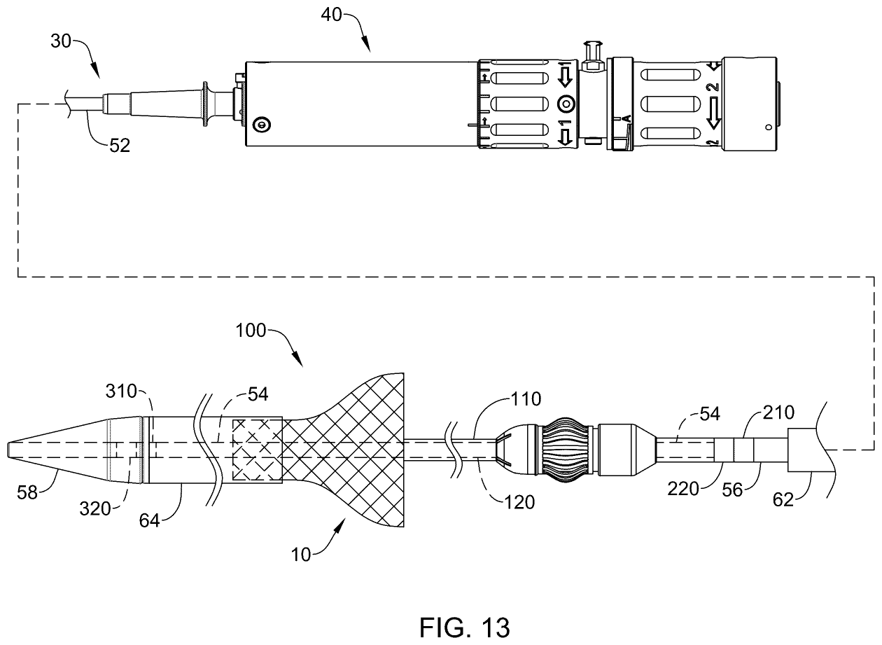

In some embodiments, the attachment assembly 200 may comprise threads, as seen schematically in FIG. 7. In some embodiments, the first portion 210 of the attachment assembly 200 may comprise male threads 211 and the second portion 220 of the attachment assembly 200 may comprise female threads 221 configured to threadably mate with the male threads of the first portion 210 of the attachment assembly 200. In some embodiments, the first portion 210 of the attachment assembly 200 may comprise female threads and the second portion 220 of the attachment assembly 200 may comprise male threads configured to threadably mate with the female threads of the first portion 210 of the attachment assembly 200. In some embodiments, the intermediate tubular member 56 may be configured to rotate relative to the first elongate shaft 110, or vice versa, to engage the first portion 210 of the attachment assembly 200 with the second portion 220 of the attachment assembly 200, as may be understood from FIGS. 12-13.

In some embodiments, the attachment assembly 300 may comprise threads, as seen schematically in FIG. 7. In some embodiments, the first portion 310 of the attachment assembly 300 may comprise male threads 311 and the second portion 320 of the attachment assembly 300 may comprise female threads 321 configured to threadably mate with the male threads of the first portion 310 of the attachment assembly 300. In some embodiments, the first portion 310 of the attachment assembly 300 may comprise female threads and the second portion 320 of the attachment assembly 300 may comprise male threads configured to threadably mate with the female threads of the first portion 310 of the attachment assembly 300. In some embodiments, the inner shaft 54 may be configured to rotate relative to the distal tip 58 and/or the distal sheath 64, or vice versa, to engage the first portion 310 of the attachment assembly 300 with the second portion 320 of the attachment assembly 300, as may be understood from FIGS. 12-13. While the second portion 320 is illustrated in FIG. 7 as being coupled to, attached to, and/or a part of a tubular member or shaft, it shall be understood that the second portion 320 may be integrated into, attached to, coupled to, etc. the distal tip 58 and/or the distal sheath 64.

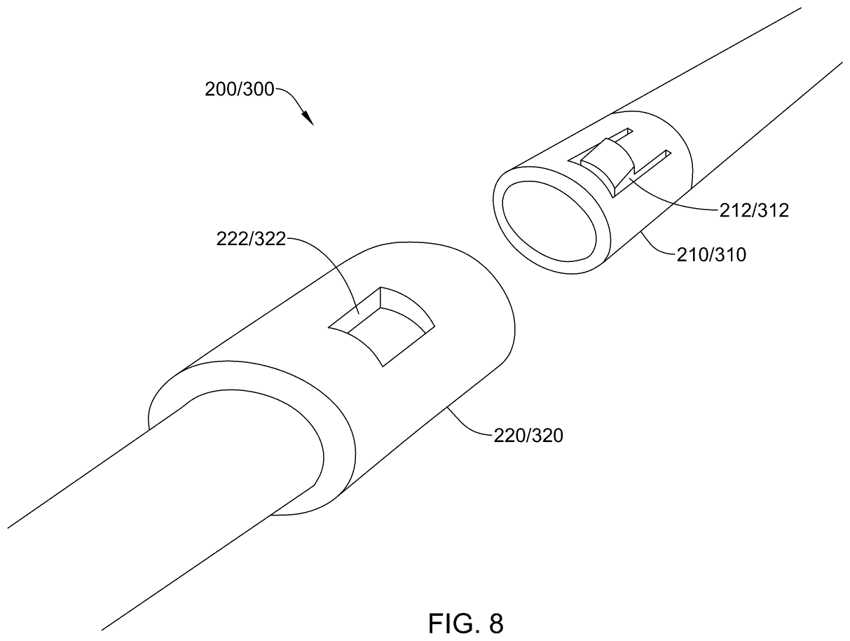

In some embodiments, the attachment assembly 200 may comprise a snap lock, as seen schematically in FIG. 8. In some embodiments, the first portion 210 of the attachment assembly 200 may comprise a biased locking element 212 and the second portion 220 of the attachment assembly 200 may comprise an aperture 222 configured to receive the biased locking element 212 of the first portion 210 of the attachment assembly 200. In some embodiments, the second portion 220 of the attachment assembly 200 may comprise the biased locking element and the first portion 210 of the attachment assembly 200 may comprise the aperture configured to receive the biased locking element of the second portion 220 of the attachment assembly 200. In some embodiments, the biased locking element 212 may be biased toward a locking position. In at least some embodiments, the biased locking element 212 may be self-biased toward the locking position. Other configurations are also contemplated. In the locking position, the biased locking element 212 may be configured to engage with the aperture 222. The biased locking element 212 may comprise a radially outermost extent at a first end, and a radially outward facing surface that tapers radially inward toward a second end of the biased locking element 212 disposed opposite the first end, wherein the second end is disposed closer to the aperture 222 than the first end when the first portion 210 is coaxially aligned with the second portion 220 and the first portion 210 is not engaged with the second portion 220. In some embodiments, the intermediate tubular member 56 may be configured to axially translate relative to the first elongate shaft 110, or vice versa, to engage the first portion 210 of the attachment assembly 200 with the second portion 220 of the attachment assembly 200, as may be understood from FIGS. 12-13. When the first portion 210 is engaged with the second portion 220, the first end of the biased locking element 212 may matingly engage with the aperture 222 to prevent the first portion 210 from disengaging from the second portion 220.

In some embodiments, the attachment assembly 300 may comprise a snap lock, as seen schematically in FIG. 8. In some embodiments, the first portion 310 of the attachment assembly 300 may comprise a biased locking element 312 and the second portion 320 of the attachment assembly 300 may comprise an aperture 322 configured to receive the biased locking element 312 of the first portion 310 of the attachment assembly 300. In some embodiments, the second portion 320 of the attachment assembly 300 may comprise the biased locking element and the first portion 310 of the attachment assembly 300 may comprise the aperture configured to receive the biased locking element of the second portion 320 of the attachment assembly 300. In some embodiments, the biased locking element 312 may be biased toward a locking position. In at least some embodiments, the biased locking element 312 may be self-biased toward the locking position. Other configurations are also contemplated. In the locking position, the biased locking element 312 may be configured to engage with the aperture 322. The biased locking element 312 may comprise a radially outermost extent at a first end, and a radially outward facing surface that tapers radially inward toward a second end of the biased locking element 312 disposed opposite the first end, wherein the second end is disposed closer to the aperture 322 than the first end when the first portion 310 is coaxially aligned with the second portion 320 and the first portion 310 is not engaged with the second portion 320. In some embodiments, the inner shaft 54 may be configured to rotate relative to the distal tip 58 and/or the distal sheath 64, or vice versa, to engage the first portion 310 of the attachment assembly 300 with the second portion 320 of the attachment assembly 300, as may be understood from FIGS. 12-13. When the first portion 310 is engaged with the second portion 320, the first end of the biased locking element 312 may matingly engage with the aperture 322 to prevent the first portion 310 from disengaging from the second portion 320. While the second portion 320 is illustrated in FIG. 8 as being coupled to, attached to, and/or a part of a tubular member or shaft, it shall be understood that the second portion 320 may be integrated into, attached to, coupled to, etc. the distal tip 58 and/or the distal sheath 64.

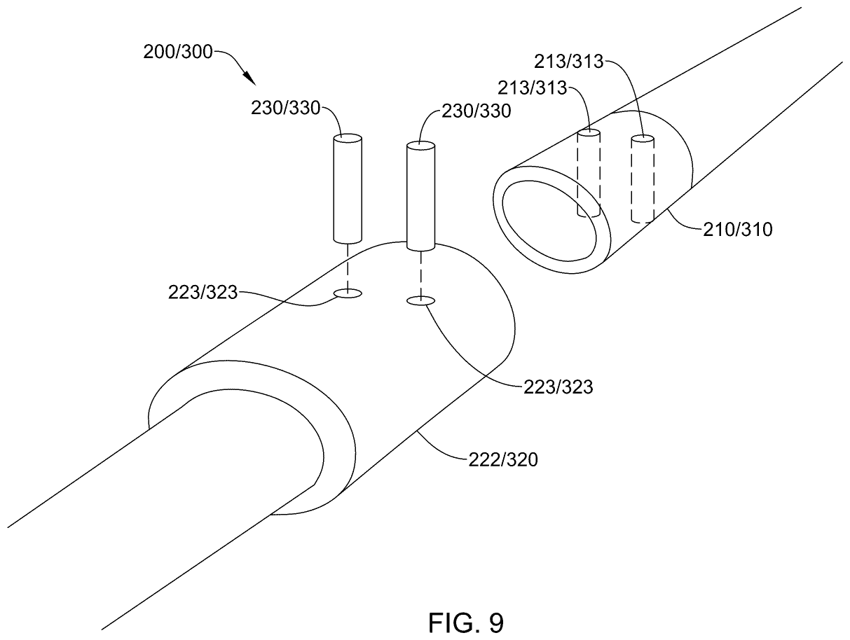

In some embodiments, the attachment assembly 200 may comprise a pin lock, as seen schematically in FIG. 9. In some embodiments, the first portion 210 of the attachment assembly 200 may comprise at least one first aperture 213 and the second portion 220 of the attachment assembly 200 may comprise at least one second aperture 223, wherein the at least one first aperture 213 is configured to align with the at least one second aperture 223 to receive at least one pin 230 of the attachment assembly 200 within the at least one first aperture 213 and the at least one second aperture 223 to lock the first portion 210 of the attachment assembly 200 to the second portion 220 of the attachment assembly 200. In some embodiments, the at least one first aperture 213 may be disposed within a side wall of the first portion 210. In some embodiments, the at least one first aperture 213 may extend through the side wall of the first portion 210. In some embodiments, the at least one first aperture 213 may be formed entirely within the side wall of the first portion 210. Other configurations are also contemplated. In some embodiments, the at least one second aperture 223 may be disposed within a side wall of the second portion 220. In some embodiments, the at least one second aperture 223 may extend through the side wall of the second portion 220. In some embodiments, the at least one second aperture 223 may be formed entirely within the side wall of the second portion 220. Other configurations are also contemplated. In some embodiments, the intermediate tubular member 56 may be configured to axially translate relative to the first elongate shaft 110, or vice versa, to align the at least one first aperture 213 of the first portion 210 of the attachment assembly 200 with the at least one second aperture 223 of the second portion 220 of the attachment assembly 200. Thereafter, the at least one pin 230 may be inserted into the at least one first aperture 213 and the at least one second aperture 223 to engage and/or lock the first portion 210 of the attachment assembly 200 to the second portion 220 of the attachment assembly 200. When the first portion 210 is engaged with and/or locked to the second portion 220, the at least one pin 230 may matingly engage with the at least one first aperture 213 and the at least one second aperture 223 to prevent the first portion 210 from disengaging from the second portion 220.

In some embodiments, the attachment assembly 300 may comprise a pin lock, as seen schematically in FIG. 9. In some embodiments, the first portion 310 of the attachment assembly 300 may comprise at least one first aperture 313 and the second portion 320 of the attachment assembly 300 may comprise at least one second aperture 323, wherein the at least one first aperture 313 is configured to align with the at least one second aperture 323 to receive at least one pin 330 of the attachment assembly 300 within the at least one first aperture 313 and the at least one second aperture 323 to lock the first portion 310 of the attachment assembly 300 to the second portion 320 of the attachment assembly 300. In some embodiments, the at least one first aperture 313 may be disposed within a side wall of the first portion 310. In some embodiments, the at least one first aperture 313 may extend through the side wall of the first portion 310. In some embodiments, the at least one first aperture 313 may be formed entirely within the side wall of the first portion 310. Other configurations are also contemplated. In some embodiments, the at least one second aperture 323 may be disposed within a side wall of the second portion 320. In some embodiments, the at least one second aperture 323 may extend through the side wall of the second portion 320. In some embodiments, the at least one second aperture 323 may be formed entirely within the side wall of the second portion 320. Other configurations are also contemplated. In some embodiments, the inner shaft 54 may be configured to rotate relative to the distal tip 58 and/or the distal sheath 64, or vice versa, to align the at least one first aperture 313 of the first portion 310 of the attachment assembly 300 with the at least one second aperture 323 of the second portion 320 of the attachment assembly 300. Thereafter, the at least one pin 330 may be inserted into the at least one first aperture 313 and the at least one second aperture 323 to engage and/or lock the first portion 310 of the attachment assembly 300 to the second portion 320 of the attachment assembly 300. When the first portion 310 is engaged with and/or locked to the second portion 320, the at least one pin 330 may matingly engage with the at least one first aperture 313 and the at least one second aperture 323 to prevent the first portion 310 from disengaging from the second portion 320. While the second portion 320 is illustrated in FIG. 9 as being coupled to, attached to, and/or a part of a tubular member or shaft, it shall be understood that the second portion 320 may be integrated into, attached to, coupled to, etc. the distal tip 58 and/or the distal sheath 64.

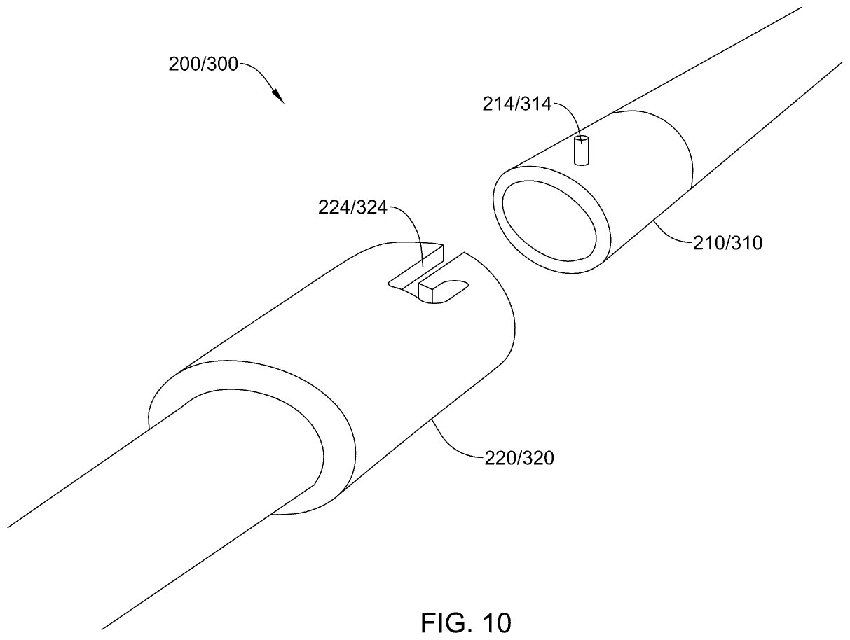

In some embodiments, the attachment assembly 200 may comprise a twist lock, as seen schematically in FIG. 10. In some embodiments, the first portion 210 of the attachment assembly 200 may comprise a pin or a projection 214 extending radially and the second portion 220 of the attachment assembly 200 may comprise a slot 224 configured to receive the pin or the projection 214 of the first portion 210 of the attachment assembly 200. In some embodiments, the second portion 220 of the attachment assembly 200 may comprise the pin or the projection and the first portion 210 of the attachment assembly 200 may comprise the slot configured to receive the pin or the projection of the second portion 220 of the attachment assembly 200. In some embodiments, the slot 224 may comprise a first axial portion open to a free end of the second portion 220 and extending axially away from the free end of the second portion to an interior end of the slot 224, a lateral portion extending circumferentially from the interior end of the first axial portion, and a second axial portion circumferentially offset from the first axial portion and extending axially from the lateral portion toward the free end of the second portion 220. The pin or the projection 214 may be configured to engage with and/or translate within the slot 224. In some embodiments, the intermediate tubular member 56 may be configured to translate relative to the first elongate shaft 110, or vice versa, to engage the first portion 210 of the attachment assembly 200 with the second portion 220 of the attachment assembly 200. In some embodiments, when the first portion 210 is engaged with the second portion 220, the pin or the projection 214 may engage with the second axial portion of the slot 224 to prevent the first portion 210 from disengaging from the second portion 220. In some embodiments, when the first portion 210 is engaged with the second portion 220, the first portion 210 may be biased away from the second portion 220 and the pin or the projection 214 may engage with the second axial portion of the slot 224 to prevent the first portion 210 from disengaging from the second portion 220.

In some embodiments, the attachment assembly 300 may comprise a twist lock, as seen schematically in FIG. 10. In some embodiments, the first portion 310 of the attachment assembly 300 may comprise a pin or a projection 314 extending radially and the second portion 320 of the attachment assembly 300 may comprise a slot 324 configured to receive the pin or the projection 314 of the first portion 310 of the attachment assembly 300. In some embodiments, the second portion 320 of the attachment assembly 300 may comprise the pin or the projection and the first portion 310 of the attachment assembly 300 may comprise the slot configured to receive the pin or the projection of the second portion 320 of the attachment assembly 300. In some embodiments, the slot 324 may comprise a first axial portion open to a free end of the second portion 320 and extending axially away from the free end of the second portion to an interior end of the slot 324, a lateral portion extending circumferentially from the interior end of the first axial portion, and a second axial portion circumferentially offset from the first axial portion and extending axially from the lateral portion toward the free end of the second portion 320. The pin or the projection 314 may be configured to engage with and/or translate within the slot 324. In some embodiments, the inner shaft 54 may be configured to rotate relative to the distal tip 58 and/or the distal sheath 64, or vice versa, to engage the first portion 310 of the attachment assembly 300 with the second portion 320 of the attachment assembly 300. In some embodiments, when the first portion 310 is engaged with the second portion 320, the pin or the projection 314 may engage with the second axial portion of the slot 324 to prevent the first portion 310 from disengaging from the second portion 320. In some embodiments, when the first portion 310 is engaged with the second portion 320, the first portion 310 may be biased away from the second portion 320 and the pin or the projection 314 may engage with the second axial portion of the slot 324 to prevent the first portion 310 from disengaging from the second portion 320. While the second portion 320 is illustrated in FIG. 10 as being coupled to, attached to, and/or a part of a tubular member or shaft, it shall be understood that the second portion 320 may be integrated into, attached to, coupled to, etc. the distal tip 58 and/or the distal sheath 64.

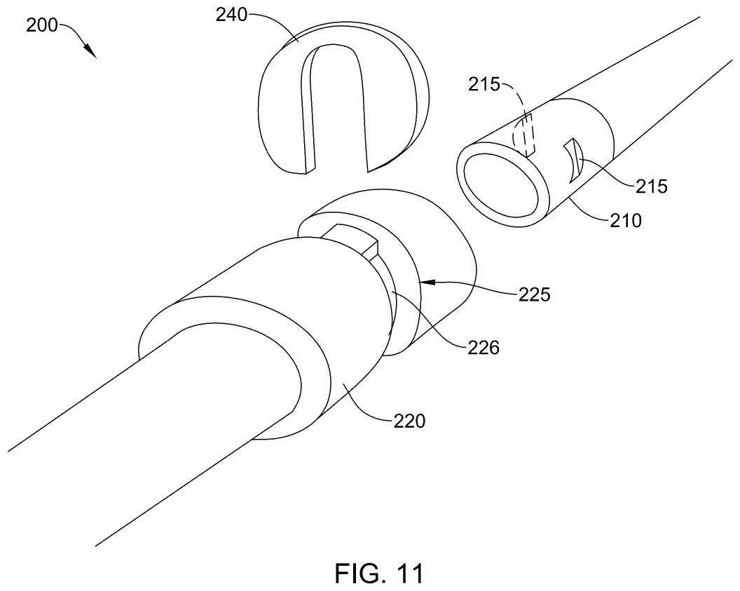

In some embodiments, the attachment assembly 200 may comprise a clip lock, as seen schematically in FIG. 11. In some embodiments, the first portion 210 of the attachment assembly 200 may comprise at least one first slot 215 formed in a side wall of the first portion 210, and the second portion 220 of the attachment assembly 200 may comprise a groove 225 extending circumferentially around the second portion 220 within a side wall of the second portion 220 and at least one second slot 226 extending through the side wall of the second portion 220, wherein the at least one first slot 215 is configured to align with the at least one second slot 226 to receive a C-clip 240 of the attachment assembly 200 within the at least one first slot 215 and the at least one second slot 226 to lock the first portion 210 of the attachment assembly 200 to the second portion 220 of the attachment assembly 200. In at least some embodiments, the groove 225 may be configured to receive the C-clip 240 therein such that a radially outer surface of the C-clip 240 may be flush with and/or recessed radially inward of an outer surface of the second portion 220. In some alternative embodiments, the at least one first slot 215 may extend through the side wall of the first portion 210. In some embodiments, the intermediate tubular member 56 may be configured to axially translate relative to the first elongate shaft 110, or vice versa, to align the at least one first slot 215 of the first portion 210 of the attachment assembly 200 with the at least one second slot 226 of the second portion 220 of the attachment assembly 200. Thereafter, the C-clip 240 may be inserted into the at least one first slot 215, the at least one second slot 226, and/or the groove 225 to engage and/or lock the first portion 210 of the attachment assembly 200 to the second portion 220 of the attachment assembly 200. When the first portion 210 is engaged with and/or locked to the second portion 220, the C-clip 240 may matingly engage with the at least one first slot 215 and the at least one second slot 226 to prevent the first portion 210 from disengaging from the second portion 220. It shall also be understood that in some embodiments, the first portion 210 and the second portion 220 as described herein with respect to FIG. 11 may be reversed and/or interchanged such that features described with respect to the first portion 210 may be found in the second portion 220 and features described with respect to the second portion 220 may be found in the first portion 210.

FIGS. 12-14 illustrate selected aspects of the replacement heart valve system and a method of loading the replacement heart valve implant 10 into the replacement heart valve system. Reference numbers for some elements are discussed in more detail with respect to other figures and are provided in FIGS. 12-14 for ease of understanding. Additionally, some features are shown schematically while other are omitted to improve clarity.

In some embodiments, the method of loading the replacement heart valve implant 10 may comprise positioning the implant delivery subassembly 100 adjacent the implant delivery system 30 in the pre-loaded configuration, as seen in FIG. 12. In at least some embodiments, the implant delivery subassembly 100 may be initially physically separate from the implant delivery system 30 while the implant delivery subassembly 100 is disposed in the pre-loaded configuration. In some embodiments, the implant delivery system 30 and the implant delivery subassembly 100 may be provided in separate packages. In some embodiments, the implant delivery subassembly 100 may be provided within a package 390 (e.g., FIGS. 5-6) that is disposed within the same package as the implant delivery system 30. In some embodiments, the package 390 may be physically separate from a package holding the implant delivery system 30. In some embodiments, positioning the implant delivery subassembly 100 adjacent the implant delivery system 30 in the pre-loaded configuration may comprise removing the implant delivery subassembly 100 from the package 390 in the pre-loaded configuration.

In some embodiments, the method of loading the replacement heart valve implant 10 may comprise coupling the implant delivery subassembly 100 to the implant delivery system 30, as seen in FIG. 13. In some embodiments, the implant delivery subassembly 100 is couplable to the implant delivery system 30 while the implant delivery subassembly is in the pre-loaded configuration. In some embodiments, the method of loading the replacement heart valve implant 10 may comprise coupling the implant delivery subassembly 100 to the implant delivery system 30 in the pre-loaded configuration.

In some embodiments, in the pre-loaded configuration of the implant delivery subassembly 100, the distal tip 58 and/or the distal sheath 64 is configured to be coupled to a distal end of the inner shaft 54 of the implant delivery system 30. In some embodiments, in the pre-loaded configuration of the implant delivery subassembly 100, the first elongate shaft 110 is configured to be coupled to the intermediate tubular member 56 of the implant delivery system 30 disposed about the inner shaft 54. In some embodiments, coupling the implant delivery subassembly 100 to the implant delivery system 30 comprises coupling the first elongate shaft 110 to the intermediate tubular member 56 of the implant delivery system 30. In some embodiments, coupling the implant delivery subassembly 100 to the implant delivery system 30 comprises extending the inner shaft 54 of the implant delivery system 30 through the lumen 120 of the first elongate shaft 110 and coupling the distal tip 58 and/or the distal sheath 64 of the implant delivery subassembly 100 to the inner shaft 54 of the implant delivery system 30. In some embodiments, the inner shaft 54 may extend distally from the handle assembly 40 within the intermediate tubular member 56 and/or the first elongate shaft 110 to the distal tip 58 and/or the distal sheath 64.

In some embodiments, coupling the implant delivery subassembly 100 to the implant delivery system 30 comprises engaging the first portion 210 of the attachment assembly 200 with the second portion 220 of the attachment assembly 200. In some embodiments, coupling the implant delivery subassembly 100 to the implant delivery system 30 comprises engaging the first portion 310 of the attachment assembly 300 with the second portion 320 of the attachment assembly 300.

In at least some embodiments, after the first portion 210 of the attachment assembly 200 is engaged with the second portion 220 of the attachment assembly 200, the first portion 210 of the attachment assembly 200 may be non-detachable from, and/or may be unable to disengage from, the second portion 220 of the attachment assembly 200 in vivo. In some embodiments, after the first portion 310 of the attachment assembly 300 is engaged with the second portion 320 of the attachment assembly 300, the first portion 310 of the attachment assembly 300 may be non-detachable from, and/or may be unable to disengage from, the second portion 320 of the attachment assembly 300 in vivo. Accordingly, in some embodiments, while the implant delivery subassembly 100 may be detachable from the implant delivery system 30 via manual manipulation and concerted effort, to replace a defective implant delivery subassembly 100 discovered prior to an implantation procedure for example, the implant delivery subassembly 100 is prevented from decoupling from the implant delivery system 30 during normal use and/or while delivering the replacement heart valve implant 10 to the native heart valve.

In some embodiments, the method of loading the replacement heart valve implant 10 may comprise, after coupling the implant delivery subassembly 100 to the implant delivery system 30, shifting the proximal sheath 62 of the implant delivery system 30 distally over the proximal portion of the replacement heart valve implant 10 to define a loaded configuration, as seen in FIG. 14. In some embodiments, after coupling the implant delivery subassembly 100 to the implant delivery system 30 while in the pre-loaded configuration, the proximal sheath 62 may be configured to be shifted distally over the proximal portion of the replacement heart valve implant 10 to define the loaded configuration.

In some embodiments, the proximal shuttle assembly 150 may include a plurality of spring elements self-biased to expand radially outward from the first elongate shaft 110, thereby exerting a radially outward force against an inner surface of the proximal sheath 62 during loading of the replacement heart valve implant 10 into the proximal sheath 62 to center the proximal sheath 62 about the first elongate shaft 110.

In some embodiments, the proximal sheath 62 and the distal sheath 64 may cooperate to and/or may collectively define an implant holding portion 60 of the replacement heart valve system. In some embodiments, the implant holding portion 60 may be configured to engage with and/or constrain the replacement heart valve implant 10 in the radially collapsed configuration when the implant holding portion 60 is disposed in a closed configuration, as seen in FIG. 14. In some embodiments, the proximal sheath 62 and/or the distal sheath 64 may be formed from a polymeric material. In some embodiments, the proximal sheath 62 and/or the distal sheath 64 may include a reinforcing structure disposed therein and/or thereon. In some embodiments, the reinforcing structure may be a coil, a mesh, one or more filaments, bands, or strips, or another suitable structure. Other configurations are also contemplated.

In some embodiments, the handle assembly 40 may be configured to manipulate and/or translate the proximal sheath 62 and/or the distal sheath 64 relative to each other using the first rotatable knob 43 and/or the second rotatable knob 44. In some embodiments, the handle assembly 40 may be configured to manipulate and/or translate the inner shaft 54 and/or the distal sheath 64 relative to the outer tubular member 52, the intermediate tubular member 56, and/or the proximal sheath 62. In some embodiments, the handle assembly 40 may be configured to manipulate and/or translate the outer tubular member 52 and/or the proximal sheath 62 relative to the inner shaft 54, the intermediate tubular member 56, and/or the distal sheath 64. In some embodiments, the handle assembly 40 may be configured to axially move the inner shaft 54 relative to the outer tubular member 52 and/or the intermediate tubular member 56. In some embodiments, the handle assembly 40 may be configured to axially move the outer tubular member 52 relative to the inner shaft 54 and/or the intermediate tubular member 56.

During delivery of the replacement heart valve implant 10 to a treatment site (e.g., the native heart valve, the aortic valve, etc.), the replacement heart valve implant 10 may be disposed at least partially within the proximal sheath 62 and the distal sheath 64 in the radially collapsed configuration when the implant holding portion 60 is disposed in the closed configuration. In some embodiments, the proximal sheath 62 may be configured to cover the proximal portion and/or the outflow end of the replacement heart valve implant 10 in the radially collapsed configuration when the implant holding portion 60 is disposed in the closed configuration, and the distal sheath 64 may be configured to cover the distal portion and/or the inflow end of the replacement heart valve implant 10 in the radially collapsed configuration when the implant holding portion 60 is disposed in the closed configuration. In some embodiments, the proximal sheath 62 may be disposed adjacent to the distal sheath 64 in the closed configuration. In some embodiments, the proximal sheath 62 may abut the distal sheath 64 in the closed configuration.

In some embodiments, the proximal sheath 62 may be axially spaced apart from the distal sheath 64 in the closed configuration. In some embodiments, the proximal sheath 62 may be axially spaced apart from the distal sheath 64 in the closed configuration by less than 20% of an overall length of the replacement heart valve implant 10. In some embodiments, the proximal sheath 62 may be axially spaced apart from the distal sheath 64 in the closed configuration by less than 15% of an overall length of the replacement heart valve implant 10. In some embodiments, the proximal sheath 62 may be axially spaced apart from the distal sheath 64 in the closed configuration by less than 10% of an overall length of the replacement heart valve implant 10. In some embodiments, the proximal sheath 62 may be axially spaced apart from the distal sheath 64 in the closed configuration by less than 5% of an overall length of the replacement heart valve implant 10. Other configurations are also contemplated.

In at least some interventions, the replacement heart valve implant 10 may be deployed within the native heart valve (e.g., the native heart valve is left in place and not excised). Alternatively, the native heart valve (e.g., the aortic valve) may be removed (such as through valvuloplasty, for example) and the replacement heart valve implant 10 may be deployed in its place as a replacement.

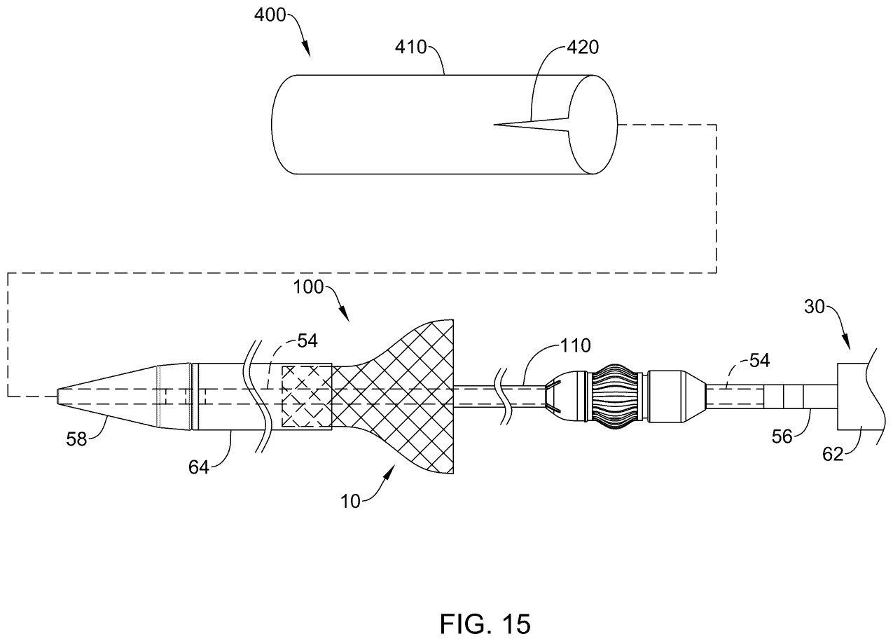

FIGS. 15-32 illustrate selected aspects of various embodiments and/or configurations of proximal loaders configured to radially collapse the proximal portion of the replacement heart valve implant 10, while coupled to the implant delivery subassembly 100 in the pre-loaded configuration, to facilitate loading the proximal portion of the replacement heart valve implant 10 within the proximal sheath 62 of the implant delivery system 30. In these figures, some reference numbers are shown to facilitate understanding but are not expressly discussed with respect to a particular figure. Additional detail(s) regarding undiscussed reference numbers may be found herein with regards to other figures.

FIG. 15 illustrates a proximal loader 400 configured to radially collapse the proximal portion of the replacement heart valve implant 10, while coupled to the implant delivery subassembly 100 in the pre-loaded configuration, to facilitate loading the proximal portion of the replacement heart valve implant 10 within the proximal sheath 62 of the implant delivery system 30. In FIG. 15, the proximal loader 400 is disposed adjacent the replacement heart valve system comprising the implant delivery system 30 and the implant delivery subassembly 100 discussed herein. The proximal loader 400 comprises a tubular sleeve 410. The tubular sleeve 410 comprises a slit 420 extending from a proximal end of the tubular sleeve 410 toward a distal end of the tubular sleeve 410. In some embodiments, the slit 420 may extend about 50% or less of a length of the tubular sleeve 410. In some embodiments, the slit 420 may extend about 40% or less of the length of the tubular sleeve 410. In some embodiments, the slit 420 may extend about 30% or less of the length of the tubular sleeve 410. In some embodiments, the slit 420 may extend about 20% or less of the length of the tubular sleeve 410. In some embodiments, the slit 420 may extend about 10% or less of the length of the tubular sleeve 410.

In some embodiments, the proximal loader 400 and/or the tubular sleeve 410 may be positioned on the implant delivery subassembly 100 and/or over the distal sheath 64 in the pre-loaded configuration. Accordingly, in such embodiments, the practitioner would not be required to position the proximal loader 400 and/or the tubular sleeve 410 on the implant delivery subassembly 100 prior to use. Importantly, in the pre-loaded configuration, the proximal loader 400 and/or the tubular sleeve 410 is not positioned over the proximal portion of the replacement heart valve implant 10 to prevent damage to the plurality of valve leaflets 20 and to facilitate sterilization. In some cases, extended compression of the plurality of valve leaflets 20 may cause creases, wrinkles, and/or stress points to form which may lead to premature failure and/or sterilization difficulties.

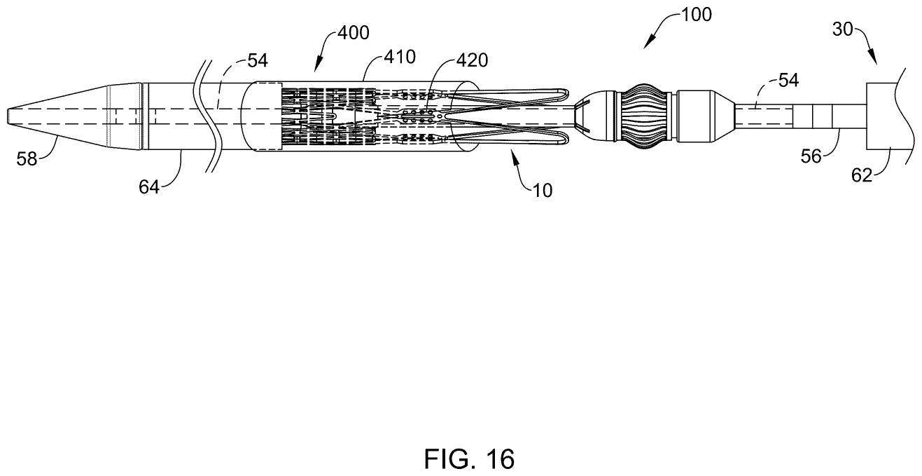

In some embodiments, after the implant delivery subassembly 100 is coupled to the implant delivery system 30, the proximal loader 400 and/or the tubular sleeve 410 may be sized and configured to slide proximally over the distal sheath 64 of the implant delivery subassembly 100 and at least a portion of the proximal portion of the replacement heart valve implant 10 to shift the proximal portion of the replacement heart valve implant 10 to the radially collapsed configuration, as seen in FIG. 16. In some embodiments, a stylet may be inserted into the distal tip 58 and/or the lumen of the inner shaft 54 to provide support during the process of loading the replacement heart valve implant 10 into the proximal sheath 62. In at least some embodiments, the slit 420 may permit the proximal end of the tubular sleeve 410 to flare radially outward slightly as the tubular sleeve 410 is slid over the distal sheath 64 of the implant delivery subassembly 100 and at least a portion of the proximal portion of the replacement heart valve implant 10. In FIG. 16, some aspects and/or portions of the replacement heart valve implant 10 are not shown to improve clarity.

In some embodiments, the proximal loader 400 and/or the tubular sleeve 410 may be formed from a polymeric material. In some embodiments, the proximal loader 400 and/or the tubular sleeve 410 may be formed from polytetrafluoroethylene (PTFE), a low-density polyethylene (LDPE), or another suitable polymer. In some embodiments, the proximal loader 400 and/or the tubular sleeve 410 may comprise a lubricious coating disposed on an inner surface thereof. Other configurations are also contemplated.

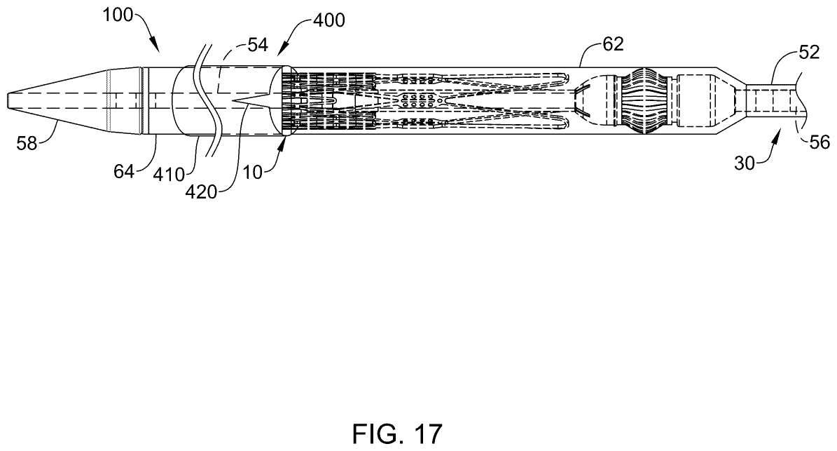

After radially collapsing the replacement heart valve implant 10 using the proximal loader 400 and/or the tubular sleeve 410, the proximal sheath 62 may be translated distally over the proximal portion of the replacement heart valve implant 10, as seen in FIG. 17, to the loaded configuration. In at least some embodiments, the one or more rotatable knobs on the handle assembly 40 may be actuated to translate the proximal sheath 62 distally relative to the intermediate tubular member 56 and/or the implant delivery subassembly 100. In some embodiments, the proximal sheath 62 may translate into and/or within the proximal loader 400 and/or the tubular sleeve 410 to capture the proximal portion of the replacement heart valve implant 10. In some embodiments, as the proximal sheath 62 is translated distally over the proximal portion of the replacement heart valve implant 10, the proximal loader 400 and/or the tubular sleeve 410 may be pushed distally by the proximal sheath 62. As such, after the proximal portion of the replacement heart valve implant 10 is disposed within the proximal sheath 62, a majority of the proximal loader 400 and/or the tubular sleeve 410 may be disposed over the distal sheath 64.

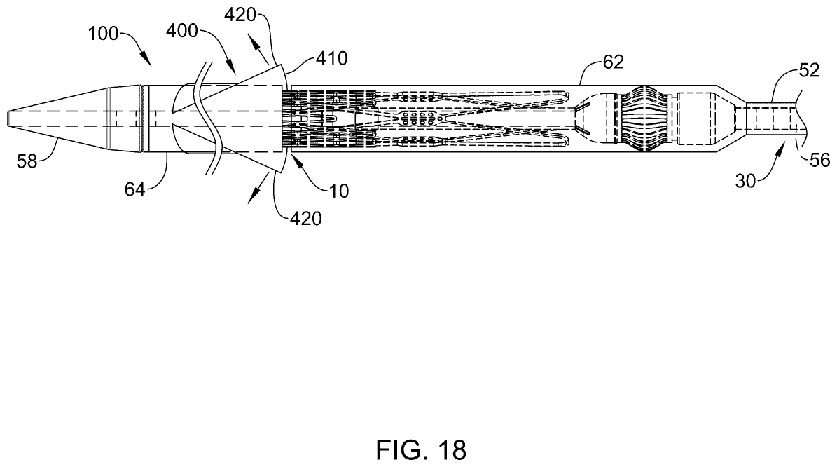

In some embodiments, after shifting and/or translating the proximal sheath 62 distally over the proximal portion of the replacement heart valve implant 10 to the loaded configuration, the proximal loader 400 and/or the tubular sleeve 410 may be removed. In some embodiments, the proximal loader 400 and/or the tubular sleeve 410 may be removable by axially translating and/or sliding the proximal loader 400 and/or the tubular sleeve 410 distally off of the implant delivery subassembly 100. In some embodiments, the proximal loader 400 and/or the tubular sleeve 410 may be removable by tearing away the proximal loader 400 and/or the tubular sleeve 410. In some embodiments, pulling on opposing sides of the slit 420 may cause the proximal loader 400 and/or the tubular sleeve 410 to split apart longitudinally along its length, as seen in FIG. 18, thereby permitting the proximal loader 400 and/or the tubular sleeve 410 to be removed without translating the proximal loader 400 and/or the tubular sleeve 410 axially. Other configurations are also contemplated.

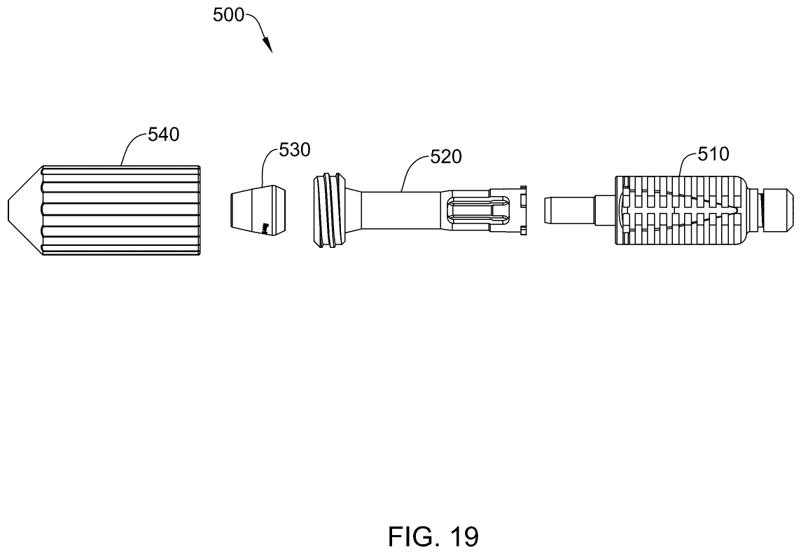

FIG. 19 is an exploded view illustrating selected aspects of a proximal loader 500 configured to radially collapse the proximal portion of the replacement heart valve implant 10, while coupled to the implant delivery subassembly 100 in the pre-loaded configuration, to facilitate loading the proximal portion of the replacement heart valve implant 10 within the proximal sheath 62 of the implant delivery system 30. The proximal loader 500 may comprise an insertion aid 510, a funnel extension 520, a crown cradle 530, and a distal cap 540.

In some embodiments, the insertion aid 510 may be releasably coupled to the funnel extension 520. In some embodiments, the insertion aid 510 and the funnel extension 520 may be disposed on the implant delivery system 30 proximate the handle assembly 40 when the implant delivery subassembly 100 is positioned adjacent the implant delivery system 30 for coupling thereto. In at least some embodiments, the insertion aid 510 and the funnel extension 520 may be disposed on the implant delivery system 30 proximate the handle assembly 40 from the manufacturer (e.g., when removed from its package). In some embodiments, the insertion aid 510 may be disposed on and/or provided on the implant delivery system 30 and the funnel extension 520 may be disposed and/or provided separately from the implant delivery system 30, wherein the funnel extension 520 may be positioned on the implant delivery system 30 and/or may be coupled to the insertion aid 510 before coupling the implant delivery subassembly 100 to the implant delivery system 30. In some embodiments, a proximal end of the funnel extension 520 may be configured to detachably couple to the insertion aid 510 using a twist lock, threads, or other suitable coupling structure.

In some embodiments, the crown cradle 530 may be configured to engage with at least a portion of the proximal portion of the replacement heart valve implant 10. In some embodiments, the crown cradle 530 may be configured to engage with at least a portion of the proximal portion of the expandable framework 12 of the replacement heart valve implant 10. In some embodiments, the crown cradle 530 may be configured to engage with the upper crown 16 of the expandable framework 12 of the replacement heart valve implant 10. In some embodiments, the crown cradle 530 may be configured to prevent radial expansion of the proximal portion of the replacement heart valve implant 10, the proximal portion of the expandable framework 12 of the replacement heart valve implant 10, and/or the upper crown 16 of the expandable framework 12 of the replacement heart valve implant 10 during loading of the replacement heart valve implant 10 into proximal sheath 62 and/or the implant holding portion 60. In some embodiments, the crown cradle 530 may be configured to prevent flaring of the distal sheath 64 during sterilization, storage of the replacement heart valve implant 10, and/or loading of the proximal portion of the replacement heart valve implant 10. In some embodiments, the crown cradle 530 may be configured to prevent the expandable framework 12 of the replacement heart valve implant 10, which may be self-expanding, from applying force to the distal sheath 64 that may cause movement of the distal sheath 64 relative to the replacement heart valve implant 10 during sterilization, storage of the replacement heart valve implant 10, and/or loading of the proximal portion of the replacement heart valve implant 10.

The distal cap 540 may be configured to threadably engage a distal end of the funnel extension 520. In at least some embodiments, the distal cap 540 may have a closed distal end. In some embodiments, the closed distal end may be tapered radially inward in a distal direction. In some embodiments, an inner surface of the closed distal end may form and/or define a distal recess configured to received and/or engage the distal tip 58 and/or a distal end of a stylet inserted into the distal tip 58. In some embodiments, the distal end of the funnel extension 520 may comprise external threads and the distal cap 540 may comprise internal threads. Other configurations are also contemplated.

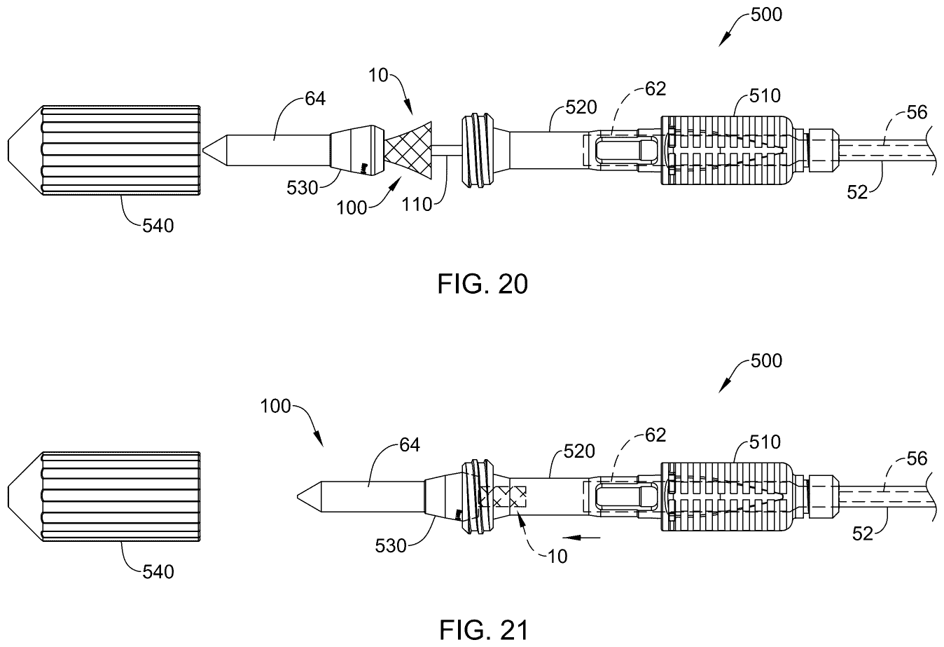

In use, after coupling the implant delivery subassembly 100 to the implant delivery system 30, the crown cradle 530 may be positioned at and/or around the upper crown 16 and/or at least a portion of the proximal portion of the replacement heart valve implant 10, which extends proximally from the distal sheath 64, as seen schematically in FIG. 20. The distal sheath 64 may extend distally from the crown cradle 530. The insertion aid 510 and the funnel extension 520 may be translated distally along the implant delivery system 30 and/or the outer tubular member 52 to a position over the proximal sheath 62, as seen schematically in FIG. 20. The insertion aid 510 may extend at least partially inside a proximal portion of the funnel extension 520 and may provide support to the proximal sheath 62 and/or the outer tubular member 52 as the replacement heart valve implant 10 is loaded into the proximal sheath 62.

Thereafter, the insertion aid 510 and the funnel extension 520, which may be coupled together and moved as a single unit and/or as a single structure, may be translated distally toward the crown cradle 530 until the distal end of the funnel extension 520 engages a proximal end of the crown cradle 530, as seen in FIG. 21. In at least some embodiments, the distal end of the funnel extension 520 may be configured to receive at least a portion of the proximal end of the crown cradle 530 therein. A distal portion of the funnel extension 520 may have an inner surface that tapers radially inward in a proximal direction to radially collapse the proximal portion of the replacement heart valve implant 10 as the funnel extension 520 and the implant delivery subassembly 100 (and/or the replacement heart valve implant 10) are translated axially relative to each other. In at least some embodiments, the insertion aid 510 may be prevented from passing over the proximal sheath 62 due to interference. In at least some embodiments, the proximal sheath 62 may have an outer diameter that is greater than an outer diameter of the outer tubular member 52 and/or is greater than an inner diameter of the proximal lumen of the insertion aid 510. In some embodiments, an attachment point between the proximal sheath 62 and the outer tubular member 52 may form an interference fit with a proximal lumen through the insertion aid 510. Other configurations are also contemplated.

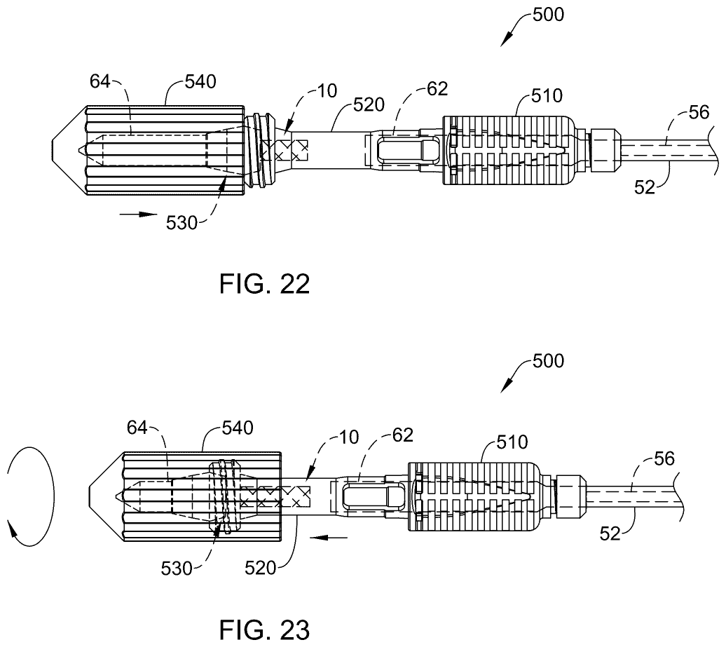

After engaging the funnel extension 520 with the crown cradle 530, the distal cap 540 may be translated proximally over the distal tip 58, the distal sheath 64, and/or the stylet disposed within the distal tip 58 to engage with the distal end of the funnel extension 520, as seen in FIG. 22. The distal tip 58 and/or the distal end of the stylet disposed within the distal tip 58 may be seated within the distal recess of the distal cap 540.

Next, the distal cap 540 may be rotated relative to the funnel extension 520 such that the internal threads within the distal cap 540 threadably engage with the external threads on the distal end of the funnel extension 520 to translate the funnel extension 520 distally relative to the distal cap 540 and/or implant delivery subassembly 100, as seen in FIG. 23. Thereafter, the proximal sheath 62 may be translated distally within the funnel extension 520 and/or relative to the implant delivery subassembly 100 to capture the proximal portion of the replacement heart valve implant 10 in the loaded configuration. In some embodiments, the proximal sheath 62 may be translated distally within the funnel extension 520 and/or relative to the implant delivery subassembly 100 via rotation of the one or more rotatable knobs on the handle assembly 40.

In at least some embodiments, the distal cap 540 may be rotated relative to the funnel extension 520 to translate the funnel extension 520 distally relative to the distal cap 540 and/or implant delivery subassembly 100 a first distance, and then the proximal sheath 62 may be translated distally within the funnel extension 520 and/or relative to the implant delivery subassembly 100 a second distance, in alternating steps, until the proximal portion of the replacement heart valve implant 10 is captured within the proximal sheath 62. In some embodiments, the first distance and the second distance may be similar. In some embodiments, the first distance and the second distance may be the same. In some embodiments, the first distance and the second distance may be commensurate with each other. In some embodiments, the first distance and the second distance may be different.

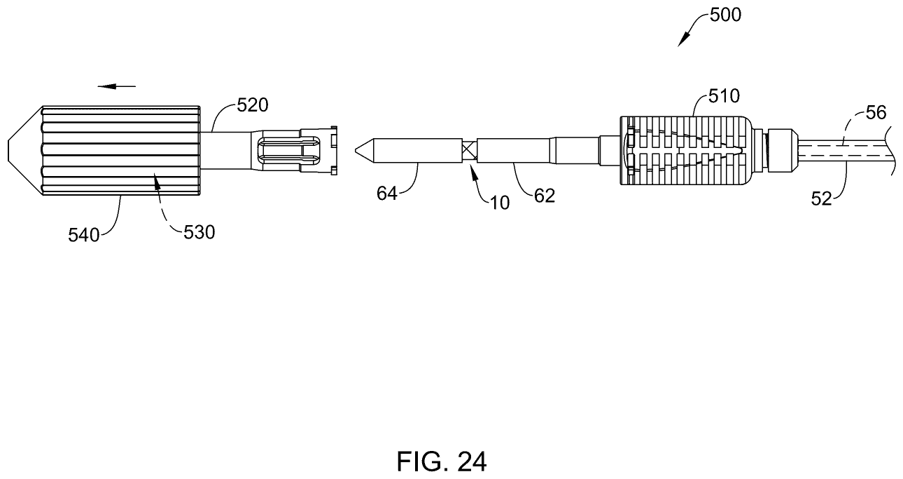

After the replacement heart valve implant 10 is disposed within the proximal sheath 62 and the distal sheath 64 in the loaded configuration (e.g., captured within the implant holding portion 60), the funnel extension 520 may be disengaged from and/or detached from the insertion aid 510 and translated distally over the distal sheath 64 and removed, as seen in FIG. 24. In at least some embodiments, the distal cap 540 may remain threadably coupled to the funnel extension 520 with the crown cradle 530 disposed therein. As such, the funnel extension 520, the crown cradle 530, and the distal cap 540 may be removed as an assembly in one distal motion after the funnel extension 520 has been disengaged from and/or detached from the insertion aid 510. In at least some embodiments, after the funnel extension 520 has been disengaged from and/or detached from the insertion aid 510, the insertion aid 510 may be translated proximally over the outer tubular member 52 and secured to the handle assembly 40, where it may remain for the duration of the procedure.

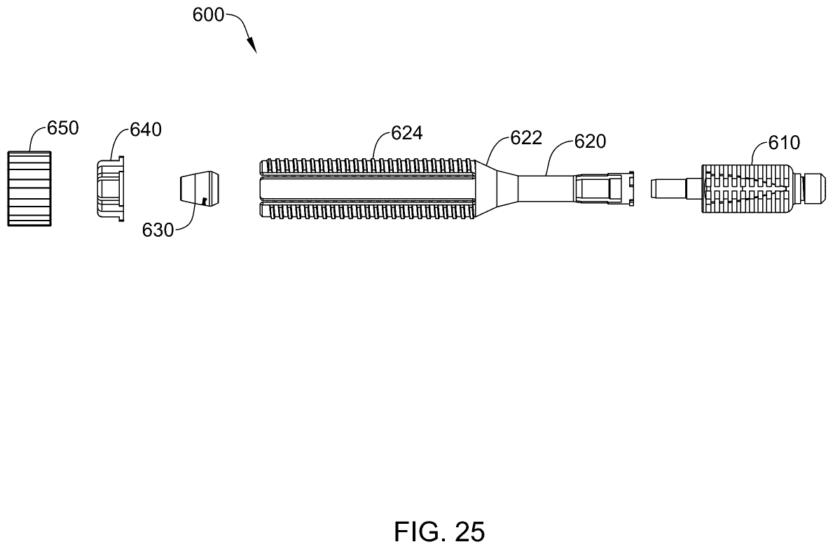

FIG. 25 is an exploded view illustrating selected aspects of a proximal loader 600 configured to radially collapse the proximal portion of the replacement heart valve implant 10, while coupled to the implant delivery subassembly 100 in the pre-loaded configuration, to facilitate loading the proximal portion of the replacement heart valve implant 10 within the proximal sheath 62 of the implant delivery system 30. The proximal loader 600 may comprise an insertion aid 610, a funnel extension 620, a crown cradle 630, a distal seat 640, and a distal cap 650.

In some embodiments, the insertion aid 610 may be releasably coupled to the funnel extension 620. In some embodiments, the insertion aid 610 and the funnel extension 620 may be disposed on the implant delivery system 30 proximate the handle assembly 40 when the implant delivery subassembly 100 is positioned adjacent the implant delivery system 30 for coupling thereto. In at least some embodiments, the insertion aid 610 and the funnel extension 620 may be disposed on the implant delivery system 30 proximate the handle assembly 40 from the manufacturer (e.g., when removed from its package). In some embodiments, the insertion aid 610 may be disposed on and/or provided on the implant delivery system 30 and the funnel extension 620 may be disposed and/or provided separately from the implant delivery system 30, wherein the funnel extension 620 may be positioned on the implant delivery system 30 and/or may be coupled to the insertion aid 610 before coupling the implant delivery subassembly 100 to the implant delivery system 30. In some embodiments, a proximal end of the funnel extension 620 may be configured to detachably couple to the insertion aid 610 using a twist lock, threads, or other suitable coupling structure. In some embodiments, the funnel extension 620 may comprise a tapered portion 622 disposed proximate a medial portion of the funnel extension 620 and a plurality of threaded fingers 624 extending distally therefrom.

In some embodiments, the crown cradle 630 may be configured to engage with at least a portion of the proximal portion of the replacement heart valve implant 10. In some embodiments, the crown cradle 630 may be configured to engage with at least a portion of the proximal portion of the expandable framework 12 of the replacement heart valve implant 10. In some embodiments, the crown cradle 630 may be configured to engage with the upper crown 16 of the expandable framework 12 of the replacement heart valve implant 10. In some embodiments, the crown cradle 630 may be configured to prevent radial expansion of the proximal portion of the replacement heart valve implant 10, the proximal portion of the expandable framework 12 of the replacement heart valve implant 10, and/or the upper crown 16 of the expandable framework 12 of the replacement heart valve implant 10 during loading of the replacement heart valve implant 10 into proximal sheath 62 and/or the implant holding portion 60. In some embodiments, the crown cradle 630 may be configured to prevent flaring of the distal sheath 64 during sterilization, storage of the replacement heart valve implant 10, and/or loading of the proximal portion of the replacement heart valve implant 10. In some embodiments, the crown cradle 630 may be configured to prevent the expandable framework 12 of the replacement heart valve implant 10, which may be self-expanding, from applying force to the distal sheath 64 that may cause movement of the distal sheath 64 relative to the replacement heart valve implant 10 during sterilization, storage of the replacement heart valve implant 10, and/or loading of the proximal portion of the replacement heart valve implant 10.

The distal cap 650 may be configured to threadably engage the plurality of threaded fingers 624 of the funnel extension 620. In some embodiments, the plurality of threaded fingers 624 of the funnel extension 620 may comprise external threads and the distal cap 650 may comprise internal threads. In at least some embodiments, the distal cap 650 may have an open distal end, such that the plurality of threaded fingers 624 of the funnel extension 620 may extend therethrough as the distal cap 650 is rotated relative to the plurality of threaded fingers 624 of the funnel extension 620 to translate the distal cap 650 relative to the funnel extension 620, the plurality of threaded fingers 624, and/or the tapered portion 622.

In some embodiments, the distal seat 640 may comprise a closed distal end tapered radially inward in a distal direction. In some embodiments, an inner surface of the closed distal end may form and/or define a distal recess configured to received and/or engage the distal tip 58 and/or a distal end of a stylet inserted into the distal tip 58. Other configurations are also contemplated. The distal seat 640 may be sized and configured to be received between the plurality of threaded fingers 624. The distal seat 640 may be configured to non-rotatably engage with the plurality of threaded fingers 624. The distal seat 640 may be received within the distal cap 650. Accordingly, as the distal cap 650 is rotated relative to the plurality of threaded fingers 624 and/or the funnel extension 620, the distal seat 640 may be prevented from rotating relative to the plurality of threaded fingers 624 and/or the funnel extension 620 while being translated axially along the plurality of threaded fingers 624.

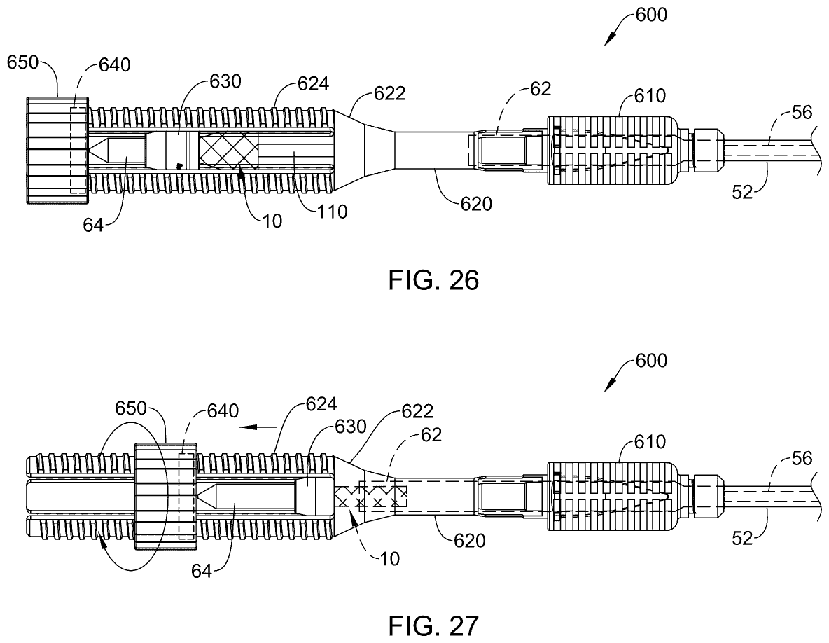

The proximal loader 600 may be used in two different ways. In a first example, after coupling the implant delivery subassembly 100 to the implant delivery system 30, the crown cradle 630 may be positioned at and/or around the upper crown 16 and/or at least a portion of the proximal portion of the replacement heart valve implant 10, which extends proximally from the distal sheath 64, as seen schematically in FIG. 26. The distal sheath 64 may extend distally from the crown cradle 630. The insertion aid 610 and the funnel extension 620, which may be coupled together and moved as a single unit and/or as a single structure, may be translated distally along the implant delivery system 30 and/or the outer tubular member 52 to a position over the proximal sheath 62. The insertion aid 610 may extend at least partially inside a proximal portion of the funnel extension 620 and may provide support to the proximal sheath 62 and/or the outer tubular member 52 as the replacement heart valve implant 10 is loaded into the proximal sheath 62. In at least some embodiments, the insertion aid 610 may be prevented from passing over the proximal sheath 62 due to interference. In at least some embodiments, the proximal sheath 62 may have an outer diameter that is greater than an outer diameter of the outer tubular member 52 and/or is greater than an inner diameter of the proximal lumen of the insertion aid 610. In some embodiments, an attachment point between the proximal sheath 62 and the outer tubular member 52 may form an interference fit with a proximal lumen through the insertion aid 610. Other configurations are also contemplated.

Next, the distal seat 640 and the distal cap 650 may be engaged with the plurality of threaded fingers 624, and the distal cap 650 may be rotated relative to the plurality of threaded fingers 624 to position the distal tip 58 and/or the distal end of the stylet disposed within the distal tip 58 in the distal recess and/or against the distal seat 640, as seen in FIG. 26. Further rotation of the distal cap 650 relative to the plurality of threaded fingers 624 may translate funnel extension 620 toward the distal seat 640 and/or relative to the implant delivery subassembly 100, as seen in FIG. 27. In at least some embodiments, the tapered portion 622 of the funnel extension 620 may be configured to receive at least a portion of the proximal end of the crown cradle 630 therein. The tapered portion 622 of the funnel extension 620 may have an inner surface that tapers radially inward in a proximal direction to radially collapse the proximal portion of the replacement heart valve implant 10 as the funnel extension 620 and the implant delivery subassembly 100 (and/or the replacement heart valve implant 10) are translated axially relative to each other.

Thereafter, the proximal sheath 62 may be translated distally within the funnel extension 620 and/or relative to the implant delivery subassembly 100 to capture the proximal portion of the replacement heart valve implant 10 in the loaded configuration. In some embodiments, the proximal sheath 62 may be translated distally within the funnel extension 620 and/or relative to the implant delivery subassembly 100 via rotation of the one or more rotatable knobs on the handle assembly 40.

In at least some embodiments, the distal cap 650 may be rotated relative to the funnel extension 620 to translate the funnel extension 620 distally relative to the distal cap 650 and/or the implant delivery subassembly 100 a first distance, and then the proximal sheath 62 may be translated distally within the funnel extension 620 and/or relative to the implant delivery subassembly 100 a second distance, in alternating steps, until the proximal portion of the replacement heart valve implant 10 is captured within the proximal sheath 62. In some embodiments, the first distance and the second distance may be similar. In some embodiments, the first distance and the second distance may be the same. In some embodiments, the first distance and the second distance may be commensurate with each other. In some embodiments, the first distance and the second distance may be different.

In a second example, the implant delivery subassembly 100 may be coupled to the implant delivery system 30 including the replacement heart valve implant 10 in the pre-loaded configuration from the manufacturer as a complete delivery system. The proximal loader 600 may be provided on the complete delivery system fully assembled (e.g., as shown in FIG. 26, and as described above with respect thereto). In some embodiments, the plurality of threaded fingers 624 may facilitate sterilization of the replacement heart valve implant 10 in the pre-loaded configuration.

After sterilization, the distal cap 650 may be rotated relative to the plurality of threaded fingers 624 to translate funnel extension 620 toward the distal seat 640 and/or relative to the implant delivery subassembly 100, as seen in FIG. 27. In at least some embodiments, the tapered portion 622 of the funnel extension 620 may be configured to receive at least a portion of the proximal end of the crown cradle 630 therein. The tapered portion 622 of the funnel extension 620 may have an inner surface that tapers radially inward in a proximal direction to radially collapse the proximal portion of the replacement heart valve implant 10 as the funnel extension 620 and the implant delivery subassembly 100 (and/or the replacement heart valve implant 10) are translated axially relative to each other.

Thereafter, the proximal sheath 62 may be translated distally within the funnel extension 620 and/or relative to the implant delivery subassembly 100 to capture the proximal portion of the replacement heart valve implant 10 in the loaded configuration. In some embodiments, the proximal sheath 62 may be translated distally within the funnel extension 620 and/or relative to the implant delivery subassembly 100 via rotation of the one or more rotatable knobs on the handle assembly 40.

In at least some embodiments, the distal cap 650 may be rotated relative to the funnel extension 620 to translate the funnel extension 620 distally relative to the distal cap 650 and/or implant delivery subassembly 100 a first distance, and then the proximal sheath 62 may be translated distally within the funnel extension 620 and/or relative to the implant delivery subassembly 100 a second distance, in alternating steps, until the proximal portion of the replacement heart valve implant 10 is captured within the proximal sheath 62. In some embodiments, the first distance and the second distance may be similar. In some embodiments, the first distance and the second distance may be the same. In some embodiments, the first distance and the second distance may be commensurate with each other. In some embodiments, the first distance and the second distance may be different.

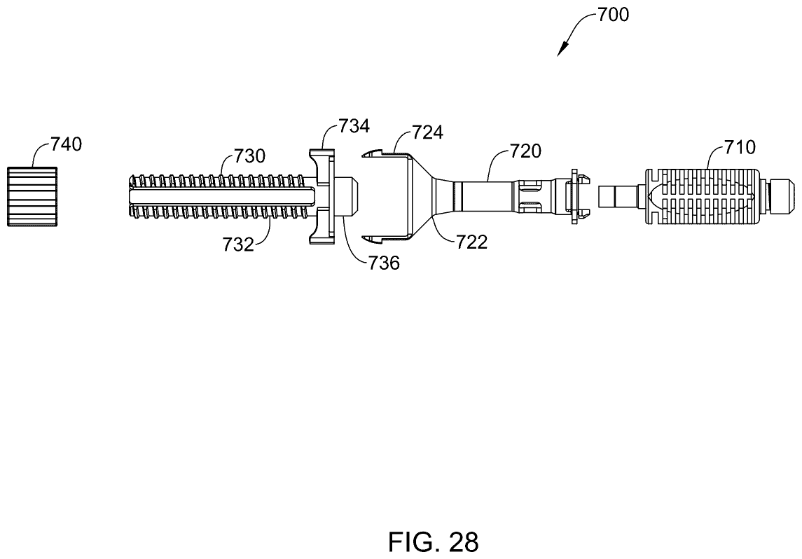

In both examples, after the replacement heart valve implant 10 is disposed within the proximal sheath 62 and the distal sheath 64 in the loaded configuration (e.g., captured within the implant holding portion 60), the funnel extension 620 may be disengaged from and/or detached from the insertion aid 610 and translated distally over the distal sheath 64 and removed, similar to the embodiment shown in FIG. 24. In at least some embodiments, the distal cap 650 may remain threadably coupled to the funnel extension 620 with the crown cradle 630 and the distal seat 640 disposed therein. As such, the funnel extension 620, the crown cradle 630, the distal seat 640, and the distal cap 650 may be removed as an assembly in one distal motion after the funnel extension 620 has been disengaged from and/or detached from the insertion aid 610. In at least some embodiments, after the funnel extension 620 has been disengaged from and/or detached from the insertion aid 610, the insertion aid 610 may be translated proximally over the outer tubular member 52 and secured to the handle assembly 40, where it may remain for the duration of the procedure. FIG. 28 is an exploded view illustrating selected aspects of a proximal loader 700 configured to radially collapse the proximal portion of the replacement heart valve implant 10, while coupled to the implant delivery subassembly 100 in the pre-loaded configuration, to facilitate loading the proximal portion of the replacement heart valve implant 10 within the proximal sheath 62 of the implant delivery system 30. The proximal loader 700 may comprise an insertion aid 710, a proximal funnel extension 720, a distal funnel extension 730, and a distal cap 740.

In some embodiments, the insertion aid 710 may be releasably coupled to the proximal funnel extension 720. In some embodiments, the insertion aid 710 and the proximal funnel extension 720 may be disposed on the implant delivery system 30 proximate the handle assembly 40 when the implant delivery subassembly 100 is positioned adjacent the implant delivery system 30 for coupling thereto. In at least some embodiments, the insertion aid 710 and the proximal funnel extension 720 may be disposed on the implant delivery system 30 proximate the handle assembly 40 from the manufacturer (e.g., when removed from its package). In some embodiments, the insertion aid 710 may be disposed on and/or provided on the implant delivery system 30 and the proximal funnel extension 720 may be disposed and/or provided separately from the implant delivery system 30, wherein the proximal funnel extension 720 may be positioned on the implant delivery system 30 and/or may be coupled to the insertion aid 710 before coupling the implant delivery subassembly 100 to the implant delivery system 30. In some embodiments, a proximal end of the proximal funnel extension 720 may be configured to detachably couple to the insertion aid 710 using a twist lock, threads, or other suitable coupling structure. In some embodiments, the proximal funnel extension 720 may comprise a tapered portion 722 disposed proximate a distal end of the proximal funnel extension 720 and a plurality of attachment arms 724 extending distally therefrom.

The distal funnel extension 730 may be couplable to the proximal funnel extension 720. In some embodiments, the distal funnel extension 730 may comprise a plurality of threaded fingers 732 extending distally from a proximal end of the distal funnel extension 730. In some embodiments, the distal funnel extension 730 may comprise a plurality of attachment sockets 734 configured to receive the plurality of attachment arms 724 of the proximal funnel extension 720. In some embodiments, the distal funnel extension 730 may comprise a crown cradle 736 integrally formed with and/or built-in to the distal funnel extension 730 at the proximal end of the distal funnel extension 730.

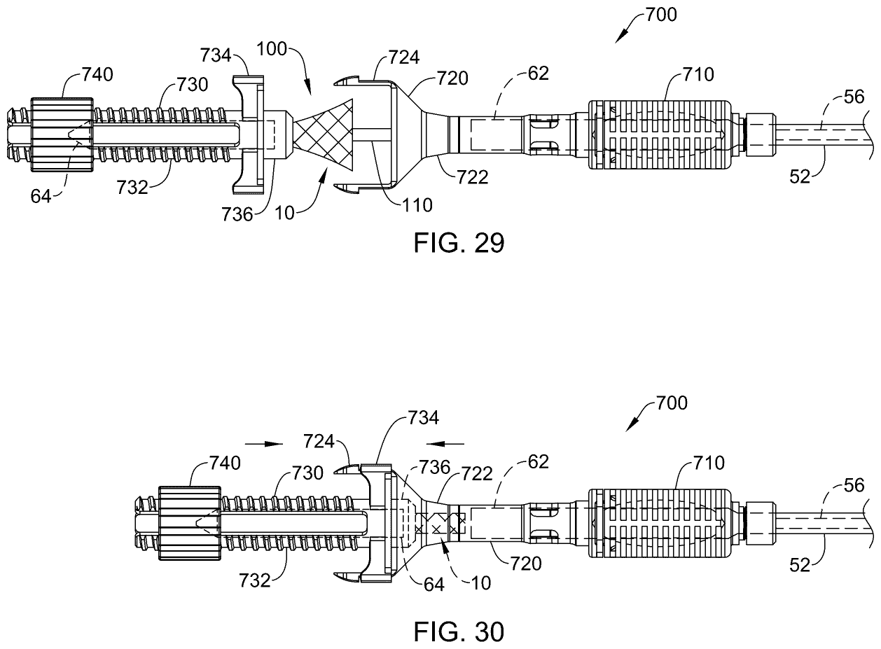

In some embodiments, the crown cradle 736 may be configured to engage with at least a portion of the proximal portion of the replacement heart valve implant 10 (e.g., FIG. 29). In some embodiments, the crown cradle 736 may be configured to engage with at least a portion of the proximal portion of the expandable framework 12 of the replacement heart valve implant 10. In some embodiments, the crown cradle 736 may be configured to engage with the upper crown 16 of the expandable framework 12 of the replacement heart valve implant 10. In some embodiments, the crown cradle 736 may be configured to prevent radial expansion of the proximal portion of the replacement heart valve implant 10, the proximal portion of the expandable framework 12 of the replacement heart valve implant 10, and/or the upper crown 16 of the expandable framework 12 of the replacement heart valve implant 10 during loading of the replacement heart valve implant 10 into proximal sheath 62 and/or the implant holding portion 60. In some embodiments, the crown cradle 736 may be configured to prevent flaring of the distal sheath 64 during sterilization, storage of the replacement heart valve implant 10, and/or loading of the proximal portion of the replacement heart valve implant 10. In some embodiments, the crown cradle 736 may be configured to prevent the expandable framework 12 of the replacement heart valve implant 10, which may be self-expanding, from applying force to the distal sheath 64 that may cause movement of the distal sheath 64 relative to the replacement heart valve implant 10 during sterilization, storage of the replacement heart valve implant 10, and/or loading of the proximal portion of the replacement heart valve implant 10.

The distal cap 740 may be configured to threadably engage the plurality of threaded fingers 732 of the distal funnel extension 730. In some embodiments, the plurality of threaded fingers 732 of the distal funnel extension 730 may comprise external threads and the distal cap 740 may comprise internal threads. In at least some embodiments, the distal cap 740 may have an open distal end, such that the plurality of threaded fingers 732 of the distal funnel extension 730 may extend therethrough as the distal cap 740 is rotated relative to the plurality of threaded fingers 732 of the distal funnel extension 730 to translate the distal cap 740 relative to the distal funnel extension 730, the plurality of threaded fingers 732, etc.

In some embodiments, the distal cap 740 may comprise an insert (not shown but may be similar to the distal seat 640 above) having a closed distal end. In some embodiments, an inner surface of the closed distal end may form and/or define a distal recess configured to received and/or engage the distal tip 58 and/or a distal end of a stylet inserted into the distal tip 58. Other configurations are also contemplated. The insert may be sized and configured to be received between the plurality of threaded fingers 732. The insert may be configured to non-rotatably engage with the plurality of threaded fingers 732. In some embodiments, as the distal cap 740 is rotated relative to the plurality of threaded fingers 732 and/or the distal funnel extension 730, the insert may be prevented from rotating relative to the plurality of threaded fingers 732 and/or the distal funnel extension 730 while being translated axially along the plurality of threaded fingers 732.

In use, after coupling the implant delivery subassembly 100 to the implant delivery system 30, the crown cradle 736 may be positioned at and/or around the upper crown 16 and/or at least a portion of the proximal portion of the replacement heart valve implant 10, which extends proximally from the distal sheath 64, as seen schematically in FIG. 29. The distal sheath 64 may extend distally from the crown cradle 736. The insertion aid 710 and the proximal funnel extension 720, which may be coupled together and moved as a single unit and/or as a single structure, may be translated distally along the implant delivery system 30 and/or the outer tubular member 52 to a position over the proximal sheath 62. The insertion aid 710 may extend at least partially inside a proximal portion of the proximal funnel extension 720 and may provide support to the proximal sheath 62 and/or the outer tubular member 52 as the replacement heart valve implant 10 is loaded into the proximal sheath 62. In at least some embodiments, the insertion aid 710 may be prevented from passing over the proximal sheath 62 due to interference. In at least some embodiments, the proximal sheath 62 may have an outer diameter that is greater than an outer diameter of the outer tubular member 52 and/or is greater than an inner diameter of the proximal lumen of the insertion aid 710. In some embodiments, an attachment point between the proximal sheath 62 and the outer tubular member 52 may form an interference fit with a proximal lumen through the insertion aid 710. Other configurations are also contemplated.

Next, the distal cap 740 and the insert may be engaged with the plurality of threaded fingers 732, and the distal cap 740 may be rotated relative to the plurality of threaded fingers 732 to position the distal tip 58 and/or the distal end of the stylet disposed within the distal tip 58 in the distal recess and/or against the distal seat 640, as seen in FIG. 29. Then, the proximal funnel extension 720 and the distal funnel extension 730 may be coupled together by engaging the plurality of attachment arms 724 with the plurality of attachment sockets 734, as seen in FIG. 30. Coupling the proximal funnel extension 720 and the distal funnel extension 730 together may translate the proximal portion of the replacement heart valve implant 10 into and/or through the tapered portion 722 of the proximal funnel extension 720. The tapered portion 722 of the proximal funnel extension 720 may have an inner surface that tapers radially inward in a proximal direction to radially collapse the proximal portion of the replacement heart valve implant 10 as the funnel extension 620 and the implant delivery subassembly 100 (and/or the replacement heart valve implant 10) are translated axially relative to each other. In at least some embodiments, coupling the proximal funnel extension 720 and the distal funnel extension 730 together may position the crown cradle 736 within and/or adjacent to the tapered portion 722 of the proximal funnel extension 720.

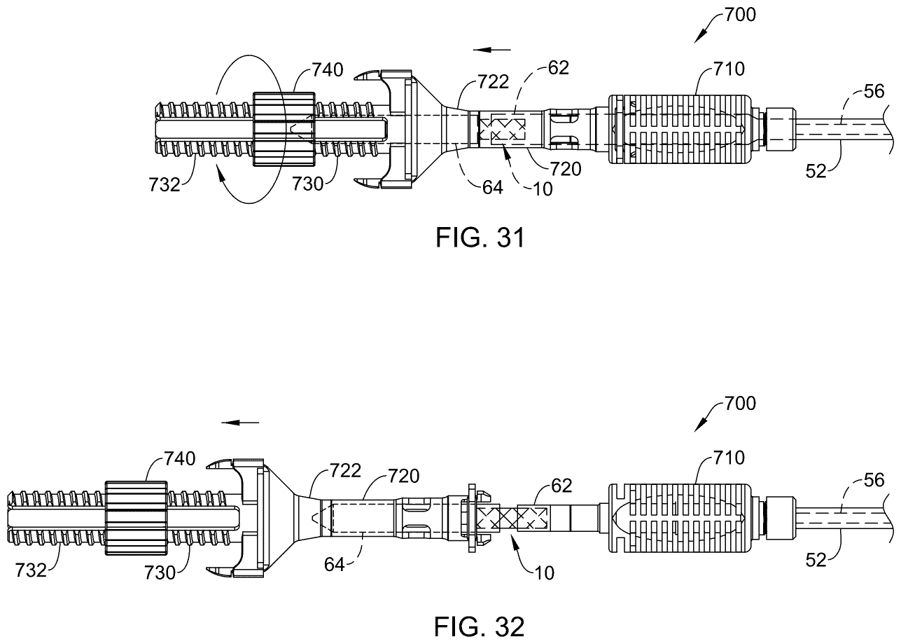

After coupling the proximal funnel extension 720 and the distal funnel extension 730 together, further rotation of the distal cap 740 relative to the plurality of threaded fingers 732 may translate the proximal funnel extension 720 toward the distal cap 740 and/or relative to the implant delivery subassembly 100, as seen in FIG. 31.

Thereafter, the proximal sheath 62 may be translated distally within the proximal funnel extension 720 and/or relative to the implant delivery subassembly 100 to capture the proximal portion of the replacement heart valve implant 10 in the loaded configuration. In some embodiments, the proximal sheath 62 may be translated distally within the proximal funnel extension 720 and/or relative to the implant delivery subassembly 100 via rotation of the one or more rotatable knobs on the handle assembly 40.

In at least some embodiments, the distal cap 740 may be rotated relative to the distal funnel extension 730 and/or the plurality of threaded fingers 732 to translate the proximal funnel extension 720 distally relative to the distal cap 740 and/or the implant delivery subassembly 100 a first distance, and then the proximal sheath 62 may be translated distally within the proximal funnel extension 720 and/or relative to the implant delivery subassembly 100 a second distance, in alternating steps, until the proximal portion of the replacement heart valve implant 10 is captured within the proximal sheath 62. In some embodiments, the first distance and the second distance may be similar. In some embodiments, the first distance and the second distance may be the same. In some embodiments, the first distance and the second distance may be commensurate with each other. In some embodiments, the first distance and the second distance may be different.

After the replacement heart valve implant 10 is disposed within the proximal sheath 62 and the distal sheath 64 in the loaded configuration (e.g., captured within the implant holding portion 60), the proximal funnel extension 720 may be disengaged from and/or detached from the insertion aid 710 and translated distally over the distal sheath 64 and removed, as seen in FIG. 32. In at least some embodiments, the distal cap 740 may remain threadably coupled to the distal funnel extension 730 with the insert disposed therein. As such, the proximal funnel extension 720, the distal funnel extension 730, the insert, and the distal cap 740 may be removed as an assembly in one distal motion after the proximal funnel extension 720 has been disengaged from and/or detached from the insertion aid 710. In at least some embodiments, after the proximal funnel extension 720 has been disengaged from and/or detached from the insertion aid 710, the insertion aid 710 may be translated proximally over the outer tubular member 52 and secured to the handle assembly 40, where it may remain for the duration of the procedure.

The materials that can be used for the various components of the replacement heart valve system and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the replacement heart valve implant, the implant delivery system, the implant delivery subassembly, the proximal loader, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni—Co—Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine);

antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A replacement heart valve system, comprising:
an implant delivery subassembly configured to be coupled to an implant delivery system, the implant delivery subassembly comprising:
a first elongate shaft comprising a lumen extending therethrough and a stent holder fixedly attached at a distal end of the first elongate shaft;
a replacement heart valve implant detachably engaged with the stent holder; and
a distal sheath disposed over a distal portion of the replacement heart valve implant and the stent holder with a proximal portion of the replacement heart valve implant disposed outside of the distal sheath to define a pre-loaded configuration;
wherein the distal sheath is configured to be coupled to a distal end of an inner shaft of the implant delivery system and the first elongate shaft is configured to be coupled to an intermediate tubular member of the implant delivery system disposed about the inner shaft.

2. The replacement heart valve system of claim 1, wherein in the pre-loaded configuration the distal sheath is configured to constrain the distal portion of the replacement heart valve implant in a radially collapsed configuration.

3. The replacement heart valve system of claim 1, wherein the lumen of the first elongate shaft is configured to slidably receive the inner shaft of the implant delivery system.

4. The replacement heart valve system of claim 1, wherein the replacement heart valve implant comprises a plurality of valve leaflets.

5. The replacement heart valve system of claim 4, wherein the implant delivery subassembly is configured to be stored in the pre-loaded configuration in a package devoid of fluid.

6. The replacement heart valve system of claim 4, wherein the implant delivery subassembly is configured to be stored in the pre-loaded configuration in a package filled with a fluid.

7. The replacement heart valve system of claim 1, wherein the implant delivery system comprises a first portion of an attachment assembly disposed at a distal end of the intermediate tubular member, the first portion of the attachment assembly being configured to engage with a second portion of the attachment assembly disposed at a proximal end of the elongate shaft of the implant delivery subassembly.

8. The replacement heart valve system of claim 7, wherein the attachment assembly comprises threads.

9. The replacement heart valve system of claim 7, wherein the attachment assembly comprises a snap lock.

10. The replacement heart valve system of claim 7, wherein the attachment assembly comprises a pin lock.

11. The replacement heart valve system of claim 7, wherein the attachment assembly comprises a twist lock.

12. The replacement heart valve system of claim 7, wherein the attachment assembly is configured to non-pivotably couple the first elongate shaft to the intermediate tubular member.

13. A replacement heart valve system, comprising:
an implant delivery system comprising an outer tubular member coupled to a proximal sheath, an intermediate tubular member slidably disposed within the outer tubular member, and an inner shaft slidably disposed within the intermediate tubular member; and
an implant delivery subassembly comprising:
a first elongate shaft comprising a lumen extending therethrough and a stent holder fixedly attached at a distal end of the first elongate shaft;
a replacement heart valve implant detachably engaged with the stent holder; and
a distal sheath disposed over a distal portion of the replacement heart valve implant and the stent holder with a proximal portion of the replacement heart valve implant disposed outside of the distal sheath to define a pre-loaded configuration;
wherein the implant delivery subassembly is initially physically separate from the implant delivery system while the implant delivery subassembly is disposed in the pre-loaded configuration.

14. The replacement heart valve system of claim 13, wherein the implant delivery subassembly is couplable to the implant delivery system while the implant delivery subassembly is disposed in the pre-loaded configuration.

15. The replacement heart valve system of claim 14, wherein the distal sheath is configured to be coupled to a distal end of the inner shaft of the implant delivery system and the first elongate shaft is configured to be coupled to the intermediate tubular member of the implant delivery system.

16. The replacement heart valve system of claim 14, wherein after coupling the implant delivery subassembly to the implant delivery system while in the pre-loaded configuration, the proximal sheath is configured to be shifted distally over the proximal portion of the replacement heart valve implant to define a loaded configuration.

17. A method of loading a replacement heart valve implant into a replacement heart valve system, comprising:
positioning an implant delivery subassembly adjacent to an implant delivery system, the implant delivery subassembly comprising:
a first elongate shaft comprising a lumen extending therethrough and a stent holder fixedly attached at a distal end of the first elongate shaft;
a replacement heart valve implant detachably engaged with the stent holder; and
a distal sheath disposed over a distal portion of the replacement heart valve implant and the stent holder with a proximal portion of the replacement heart valve implant disposed outside of the distal sheath to define a pre-loaded configuration;
coupling the implant delivery subassembly to the implant delivery system in the pre-loaded configuration; and
after coupling the implant delivery subassembly to the implant delivery system, shifting a proximal sheath of the implant delivery system distally over the proximal portion of the replacement heart valve implant to define a loaded configuration.

18. The method of claim 17, wherein coupling the implant delivery subassembly to the implant delivery system comprises coupling the first elongate shaft to an intermediate tubular member of the implant delivery system.

19. The method of claim 18, wherein coupling the implant delivery subassembly to the implant delivery system comprises extending an inner shaft of the implant delivery system through the first elongate shaft and coupling the distal sheath of implant delivery subassembly to the inner shaft of the implant delivery system.

20. The method of claim 17, further comprising, before coupling the implant delivery subassembly to the implant delivery system, removing the implant delivery subassembly from a package in the pre-loaded configuration.
